(19) United States
(12) Patent Application Publication
(10) Pub. No.: US 2010/0112061 A1
(43) Pub. Date: May 6, 2010

(54) Monophosphates as Mutual Prodrugs of Muscarinic Receptor Antagonists and Beta-Agonists for the Treatment of COPD And Chronic Bronchitis

(76) Inventors: William Baker, Bellevue, WA (US); Marcin Stasiak, Seattle, WA (US); Sundaramoorthi Swaminathan, Burlingame, CA (US); Musong Kim, Bothell, WA (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/519,311

(22) PCT Filed: Dec. 12, 2007
(86) PCT No.: PCT/US07/25375

§ 371 Date: Nov. 30, 2009

Related U.S. Application Data
(60) Provisional application No. 60/874,577, filed on Dec. 13, 2006.

(51) Int. Cl. A61K 31/661; A61K 9/14; C07F 9/145; A61K 31/675; A61K 31/665; A61P 11/08

(52) U.S. Cl. 424/484; 546/22; 546/23; 548/413; 540/542; 558/190; 549/215; 514/89; 514/80; 514/82; 514/91; 514/94; 514/79; 514/114; 514/63

(57) ABSTRACT

A mutual prodrug of a MRA and a (β-agonist for formulation for delivery by aerosolization to inhibit pulmonary bronchoconstriction is described. The mutual prodrug is preferably formulated in a small volume solution (10-500 μL) dissolved in a quarter normal saline having pH between 5.0 and 7.0 for the treatment of respiratory tract bronchoconstriction by an aerosol having mass median average diameter predominantly between 1 to 5μ, produced by nebulization or by dry powder inhaler.

### CROSS REFERENCE TO RELATED APPLICATION

This application claims the priority of U.S. Provisional Application No. 60/874,577, filed Dec. 13, 2006.

### FIELD OF THE INVENTION

The current invention relates to the preparation of novel, mutual prodrugs of muscarinic receptor antagonists (MRA) and β-agonists for delivery to the lung by aerosolization. In particular, the invention concerns the synthesis, formulation and delivery of monophosphate derivatives of MRAs as mutual MRA-β-agonist prodrugs that, when delivered to the lung, cause endogenous enzymes present in the lung tissue and airway to degrade the prodrug releasing a MRA and a β-agonist (e.g. salmeterol, albuterol) at the site of administration. The described mutual prodrugs are formulated as either liquids or dry powders and the formulation permits, and is suitable for, delivery of the prodrugs to the lung endobronchial space of airways in an aerosol having a mass median average diameter predominantly between 1 to 5μ. The formulated and delivered efficacious amount of monophosphate prodrugs is sufficient to deliver therapeutic amounts of both MRA and β-agonist for treatment of respiratory tract diseases, specifically bronchoconstriction associated with chronic bronchitis or chronic obstructive pulmonary disease (COPD).

### BACKGROUND OF THE INVENTION

The antagonists of the muscarinic receptor (particularly of the M₃ subtype) have shown therapeutic efficacy in man for the control of cholinergic tone in COPD (Witek, 1999). As often is the case for therapeutic treatments for COPD, combined pharmacologic agents are required for improved efficacy. Muscarinic receptor antagonists (MRA's; specifically M₃ antagonists) in combination with agonists of the β₂-adrenoceptor have demonstrated superior effects in treating COPD as compared to those agents administered alone (e.g. Combivent). However, even in the case of treatment with selective M₃ antagonists, significant mechanism-related side effects (mostly dry mouth, but also disturbance of ocular accommodation, reduction of GI motility, etc.) result from systemic exposure. Additionally, certain clinically proven MRA's (e.g. tiotropium) have additional strong affinity to the M2 receptor resulting in undesired cardiac side effects. Agonists of the β₂-adrenoceptor, such as albuterol or salmeterol, relax airway smooth muscles in synergistically with MRA's, however they may also lead to adverse events related to their systemic activity (e.g. tachycardia, ventricular dysrhythmias, hypokalemia).

In consideration of the aforementioned side effects it would be highly advantageous to provide a water-soluble, mutual MRA-β-agonist prodrug to mask the pharmacological properties of both agents until such a prodrug reaches lungs, is effectively delivered to the endobronchial space and is converted on-site to active drugs by the action of lung enzymes, thereby delivering a therapeutic amount of both drugs directly to the constricted tissue.

It would be advantageous to have a mutual prodrug of a MRA and a β-agonist that produces sustained release of both drugs at the site of administration. Additionally, it would be highly desirable for such a mutual prodrug to be poorly absorbed from the lung (minimizing systemic exposure) and to be sufficiently water soluble allowing flexibility in its formulation and delivery system.

It is therefore a primary object of this invention to provide novel monophospates as mutual prodrugs of a MRA and a β-agonist.

It is a further object of this invention to provide a composition of the mutual prodrugs, which is stable as a liquid or solid dosage form for nebulization or dry powder delivery. Such composition contains sufficient, but not excessive, concentration of the active substance which can be efficiently aerosolized by metered-dose inhalers, nebulization in jet, ultrasonic, pressurized, or vibrating porous plate nebulizers or by dry powder into aerosol particles predominantly within the 1 to 5μ size range, wherein the salinity and pH are adjusted to permit generation of a mutual prodrug aerosol that is well tolerated by patients, wherein the composition has an adequate shelf life.

### SUMMARY OF THE INVENTION

The present invention is directed to monophosphates as mutual prodrugs of an MRA and a β-agonist and their use and formulation for delivery by inhalation as a method to treat pulmonary bronchoconstriction. The prodrug incorporates a polar phosphate and a positively charged quaternary ammonium group or charged tertiary sulfonium group, which renders the molecule highly polar and water soluble and imparts its affinity to lung DNA and proteins thus minimizing rapid systemic absorption, as well as absorption due to swallowing. Furthermore, since the mutual prodrug cannot be activated in the absence of alkaline phosphatase, systemic side effects are eliminated due to the minimal activity of that enzyme in saliva (if the mutual prodrug gets deposited in the mouth) and low phosphatase activity in plasma, as compared to other tissues, including lungs (Testa and Mayer, 2003). Because these mutual prodrugs are of high molecular weight (some approaching 1 kDa) and contain several charged (or polar) moeities their likelihood of being absorbed if swallowed is very low. Thus, the potential for undesired oral delivery of the MRA and β-agonist is eliminated.

More specifically, the present invention is directed to a compound of the formula A

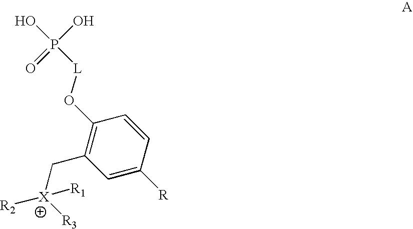

and pharmaceutical acceptable salts thereof, wherein:
X represents a quaternizable moiety, i.e. nitrogen atom, a nitrogen-containing heterocycle or a sulfur atom;
R₁R₂R₃X taken together represent either a muscarinic receptor antagonist (MRA) or its prodrug (e.g. ester) linking the parent molecule possessing MRA activity to the quaternizable moiety X, provided that when X is a sulfur atom one of R₁, R₂ and R₃ is absent:
L is a bond or methyleneoxy-(CH₂O) group; and

### R is

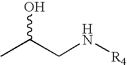

where R₄ is an alkyl group of 1-12 carbon atoms, arylalkyl or substituted arylalkyl where 1-3 CH₂ groups in the carbon chain may be replaced by atom(s) selected from O, S and NR₅ wherein R₅ is hydrogen or alkyl.

Presently preferred embodiments of this invention include compounds of formula A, wherein:

R is

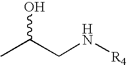

where R₄ is (CH₂)₆O(CH₂)₄Ph or tert-butyl,
L is a bond,
and R₁R₂R₃X taken together represent the muscarinic receptor antagonists:
1-{4-Hydroxy-1-[3,3,3-tris-(4-fluoro-phenyl)-propionyl]-pyrrolidine-2-carbonyl}-pyrrolidine-2-carboxylic acid (1-methyl-piperidin-4-ylmethyl)-amide;
3-[3-(2-Diethylamino-acetoxy)-2-phenyl-propionyloxy]-8-isopropyl-8-methyl-8-azonia-bicyclo[3.2.1]octane (Ipratropium-N,N-diethylglycinate);
1-Cyclohexyl-3,4-dihydro-1H-isoquinoline-2-carboxylic acid 1-aza-bicyclo[2.2.2]oct-3-yl ester (Solifenacin);
2-Hydroxymethyl-4-methanesulfinyl-2-phenyl-butyric acid 1-aza-bicyclo[2.2.2]oct-3-yl ester (Revatropate);
2-{1-[2-(2,3-Dihydro-benzofuran-5-yl)-ethyl]-pyrrolidin-3-yl}-2,2-diphenyl-acetamide (Darifenacin);
4-Azepan-1-yl-2,2-diphenyl-butyramide (Buzepide);
7-[3-(2-Diethylamino-acetoxy)-2-phenyl-propionyloxy]-9-ethyl-9-methyl-3-oxa-9-azonia-tricyclo[3.3.1.02,4]nonane (Oxitropium-N,N-diethylglycinate);
7-[2-(2-Diethylamino-acetoxy)-2,2-di-thiophen-2-yl-acetoxy]-9,9-dimethyl-3-oxa-9-azonia-tricyclo[3.3.1.02,4]nonane (Tiotropium-N,N-diethylglycinate);
Dimethylamino-acetic acid 2-(3-diisopropylamino-1-phenyl-propyl)-4-methyl-phenyl ester (Tolterodine-N,N-dimethylglycinate);
3-[4,4-Bis-(4-fluoro-phenyl)-2-oxo-imidazolidin-1-yl]-1-methyl-1-(2-oxo-2-pyridin-2-yl-ethyl)-pyrrolidinium;
1-[1-(3-Fluoro-benzyl)-piperidin-4-yl]-4,4-bis-(4-fluoro-phenyl)-imidazolidin-2-one;
1-Cyclooctyl-3-(3-methoxy-1-aza-bicyclo[2.2.2]oct-3-yl)-1-phenyl-prop-2-yn-1-ol;
3-[2-(2-Diethylamino-acetoxy)-2,2-di-thiophen-2-yl-acetoxy]-1-(3-phenoxy-propyl)-1-azonia-bicyclo[2.2.2]octane (Aclidinium-N,N-diethylglycinate); or
(2-Diethylamino-acetoxy)-di-thiophen-2-yl-acetic acid 1-methyl-1-(2-phenoxy-ethyl)-piperidin-4-yl ester.

A presently preferred embodiment of the present invention are compounds of formula B

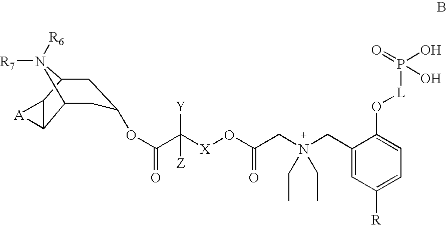

where L is a bond or CH₂—O;

R is

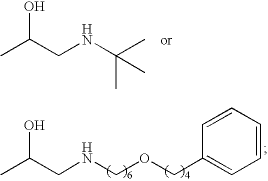

X is a bond or CH₂;

Y and Z are phenyl, 2-thienyl, or H;

R₆ is CH₃;

R₇ is ethyl, methyl or isopropyl; and

A is a bond or O.

Another presently preferred embodiment of the present invention are compounds of formula C

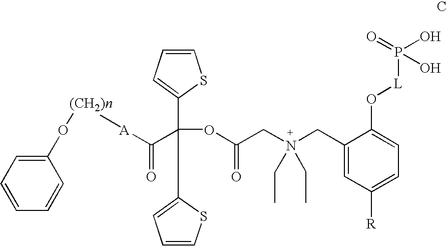

where L is a bond or CH₂—O;

R is

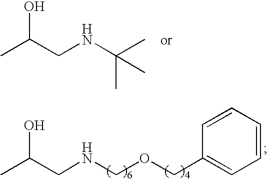

A is

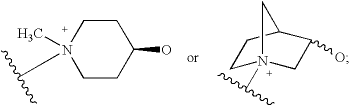

and

n is 2 or 3.

Examples of presently preferred compounds of this invention include:
Monophosphate of 3-(2-Hydroxymethyl-4-methanesulfinyl-2-phenyl-butyryloxy)-1-(5-{1-hydroxy-2-[6-(4-phenyl-butoxy)-hexylamino]-ethyl}-2-phosphonooxy-benzyl)-1-azonia-bicyclo[2.2.2]octane (Example 41);
Monophosphate of (2-methylene-4-{1-hydroxy-2-[6-(4-phenyl-butoxy)-hexylamino]-ethyl}-phenyl)-3-[3-(2-Diethylamino-acetoxy)-2-phenyl-propionyloxy]-8-isopropyl-8-methyl-8-azonia-bicyclo[3.2.1]octane (Example 33); and
Monophosphate of 3-(1-Cyclohexyl-3,4-dihydro-1H-isoquinoline-2-carbonyloxy)-1-(5-{1-hydroxy-2-[6-(4-phenyl-butoxy)-hexylamino]-ethyl}-2-phosphonooxy-benzyl)-1-azonia-bicyclo[2.2.2]octane (Example 37).

The present invention also relates to a process for the synthesis of the mutual prodrugs of formula A. The invention also relates to a pharmaceutically acceptable composition for the treatment of a disorder selected from severe to mild chronic bronchitis and COPD or other diseases related to pulmonary bronchoconstriction, which comprises a therapeutically effective amount, preferably from about 10 μg to about 1000 μg, of at least one compound of formula A, a pharmaceutically acceptable salt thereof, and a pharmaceutically acceptable carrier. The composition is preferably administered as an aerosol, most preferably by a dry powder inhaler. The invention also relates to methods of treating such diseases with therapeutically effective amounts of at least one compound of formula A or a pharmaceutically acceptable salt thereof.

The invention also relates to a liquid or dry powder formulation of the MRA-β-agonist mutual prodrug for the treatment of a disorder selected from severe to mild chronic bronchitis and COPD or other diseases related to pulmonary bronchoconstriction, which comprises a therapeutically effective amount, preferably from about 10 μg to about 1000 μg, of at least one compound of formula A or a pharmaceutically acceptable salt thereof. The composition is preferably administered as an aerosol, most preferably by a dry powder inhaler.

The invention further relates to a method for the prevention and treatment of severe to mild chronic bronchitis and COPD, comprising administering to a patient in need of such treatment an effective amount of an aerosol formulation comprising about 10 μg to about 1000 μg of the mutual prodrugs of the present invention. Preferably, when the prodrug is delivered to the lung, the phosphate group is cleaved by an endogenous enzyme alkaline phosphatase and the MRA and the β-agonist are individually released in a simultaneous manner.

### DETAILED DESCRIPTION OF THE INVENTION

As used herein, the term “aryl” is defined as a C₆-C₁₈ carbocyclic ring that may be substituted with 1-3 groups selected from hydrogen, amino, hydroxy, halo, O-alkyl and NH-alkyl. Aryl can be one or two rings either fused to form a bicylic aromatic ring system or linear such as biphenyl. One or more of the carbon atoms in an aryl group can optionally be replaced by N, S, or O in the ring to produce a heterocyclic system.

The term “alkyl” as used herein refers to a branched or straight chain comprising one to twenty carbon atoms, at least one of which can optionally be replaced one or more atoms selected from O, S, or N wherein N carries a hydrogen atom or one or more alkyl groups. Representative alkyl groups include methyl, butyl, hexyl, and the like.

As used herein, the term “lower alkyl” includes both substituted or unsubstituted straight or branched chain alkyl groups having from 1 to 10 carbon atoms. Representative lower alkyl groups include for example, methyl, ethyl, propyl, isopropyl, n-butyl, tert-butyl, and the like. Representative of halo-substituted, amino-substituted and hydroxy-substituted, lower-alkyl include chloromethyl, chloroethyl, hydroxyethyl, aminoethyl, etc.

As used herein, the term “cycloalkyl” includes a non-aromatic ring composed of 3-10 carbon atoms.

As used herein, the term “halogen” refers to chloro, bromo, fluoro and iodo groups.

The term “substituted heterocycle” or “heterocyclic group” or “heterocycle” as used herein refers to any 3- or 4-membered ring containing a heteroatom selected from nitrogen, oxygen, and sulfur or a 5- or 6-membered ring containing from one to three heteroatoms selected from the group consisting of nitrogen, oxygen, or sulfur; wherein the 5-membered ring has 0-2 double bounds and the 6-membered ring has 0-3 double bounds; wherein the nitrogen and sulfur atom may be optionally oxidized; wherein the nitrogen and sulfur heteroatoms may be optionally quarternized; and including any bicyclic group in which any of the above heterocyclic rings is fused to a benzene ring or another 5- or 6-membered heterocyclic ring independently defined above. Heterocyclics in which nitrogen is the heteroatom are preferred. Fully saturated heterocyclics are also preferred. Preferred heterocycles include: diazapinyl, pyrryl, pyrrolinyl, pyrrolidinyl, pyrazolyl, pyrazolinyl, pyrazolidinyl, imidazoyl, imidazolinyl, imidazolidinyl, pyridyl, piperidinyl, pyrazinyl, piperazinyl, azetidinyl, pyrimidinyl, pyridazinyl, oxazolyl, oxazolidinyl, isoxazolyl, isoazolidinyl, morpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, triazolyl and benzothienyl groups.

Heterocyclics can be unsubstituted or monosubstituted or disubstituted with substituents independently selected from hydroxy, halo, oxo (C═O), alkylimino (RN═, wherein R is a lower alkyl or alkoxy group), amino, alkylamino, dialkylamino, acylaminoalkyl, alkoxy, thioalkoxy, loweralkyl, cycloalkyl or haloalkyl. The most preferred heterocyclics include imidazolyl, pyridyl, piperazinyl, azetidinyl, thiazolyl, triazolyl, benzimidazolyl, benzothiazolyl and benzoxazolyl.

As used herein, the term “pharmaceutically acceptable salts” refers to a salt with a nontoxic acid or alkaline earth metal salts of the compounds of formula I. These salts can be prepared in situ during the final isolation and purification of the compounds of formula I, or separately, by reacting the base or acid functions with a suitable organic or inorganic acid or base, respectively. Representative acid salts include hydrochloride, hydrobromide, bisulfate, acetate, oxalate, valerate, oleate, palmitate, stearate, laurate, borate, benzoate, lactate, citrate, maleate, tartrate salts, and the like. Representative alkali metals of alkaline earth metal salts include sodium, potassium, calcium, and magnesium.

As used herein, the term “alkoxy” refers to —O—R wherein R is lower alkyl as defined above. Representative examples of lower alkoxy groups include methoxy, ethoxy, tert-butoxy, and the like.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

The term “normal saline” means water solution containing 0.9% (w/v) NaCl.

The term “diluted saline” means normal saline containing 0.9% (w/v) NaCl diluted into its lesser strength.

The term “quarter normal saline” or “¼ NS” means normal saline diluted to its quarter strength containing 0.225% (w/v) NaCl.

The term “prodrug” as used herein refers to a compound in which specific bond(s) of the compound are broken or cleaved by the action of an enzyme or by a biological process thereby producing or releasing a drug and compound fragment which is substantially biologically inactive.

The term “mutual prodrug” as used herein refers to a bipartite or tripartite prodrug in which specific bond(s) of the compound are broken or cleaved by the action of an enzyme or by biological process thereby producing or releasing two or more drugs or prodrugs.

Unless otherwise stated, it is understood that, whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The compounds of the invention may comprise asymmetrically substituted carbon atoms. Such asymmetrically substituted carbon atoms can result in the compounds of the invention comprising mixtures of stereoisomers at a particular asymmetrically substituted carbon atom or a single stereoisomer. As a result, racemic mixtures, mixtures of diastereomers, as well as single diastereomers of the compounds of the invention are included in the present invention. The terms “S” and “R” configuration, as used herein, are as defined by the IUPAC 1974 RECOMMENDATIONS FOR SECTION E, FUNDAMENTAL STEREOCHEMISTRY, Pure Appl. Chem. 45:13-30 (1976). The terms α and β are employed for ring positions of cyclic compounds. The α-side of the reference plane is that side on which the preferred substituent lies at the lower numbered position. Those substituents lying on the opposite side of the reference plane are assigned the β descriptor. It should be noted that this usage differs from that for cyclic stereoparents, in which “α” means “below the plane” and denotes absolute configuration. The terms α and β configuration, as used herein, are as defined by the CHEMICAL ABSTRACTS INDEX GUIDE-APPENDIX IV (1987) paragraph 203.

The present invention also relates to the processes for preparing the compounds of the invention and to the synthetic intermediates useful in such processes, as described in detail below.

### I. Preparation of the Compounds of the Invention

The compounds of the present invention can be prepared by the processes illustrated in Schemes I-VI.

A convergent route to a mutual prodrug of MRA and a β-agonist involves:

a) synthesis of the phosphorylated β-agonist derivatives activated towards alkylation (Scheme I-V); and
b) quaternization (alkylation) of the MRA molecule or its physiologically cleavable esters carrying “quaternizable moiety”, with the activated β-agonist derivative, followed by final deprotection (Scheme VI).

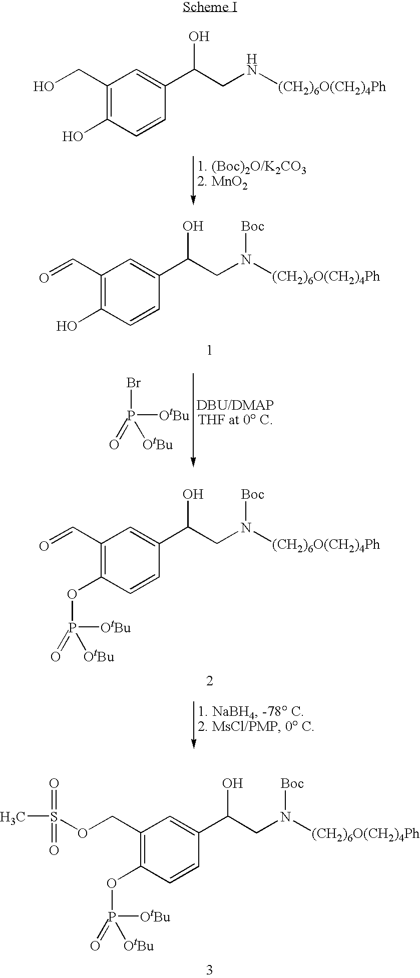

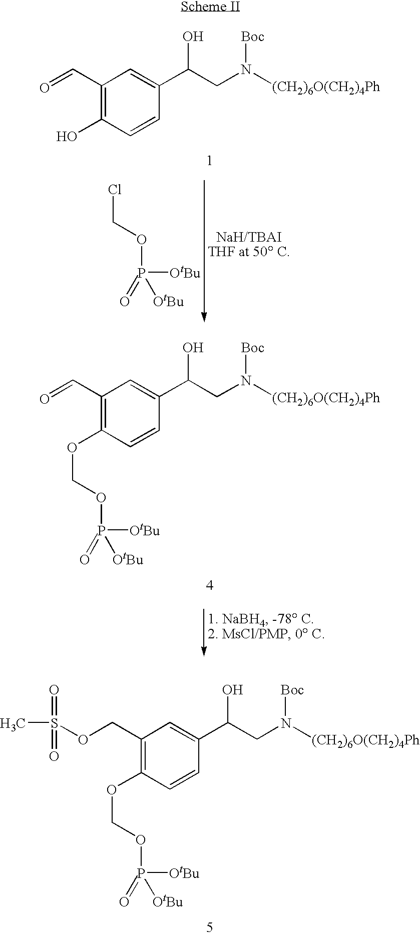

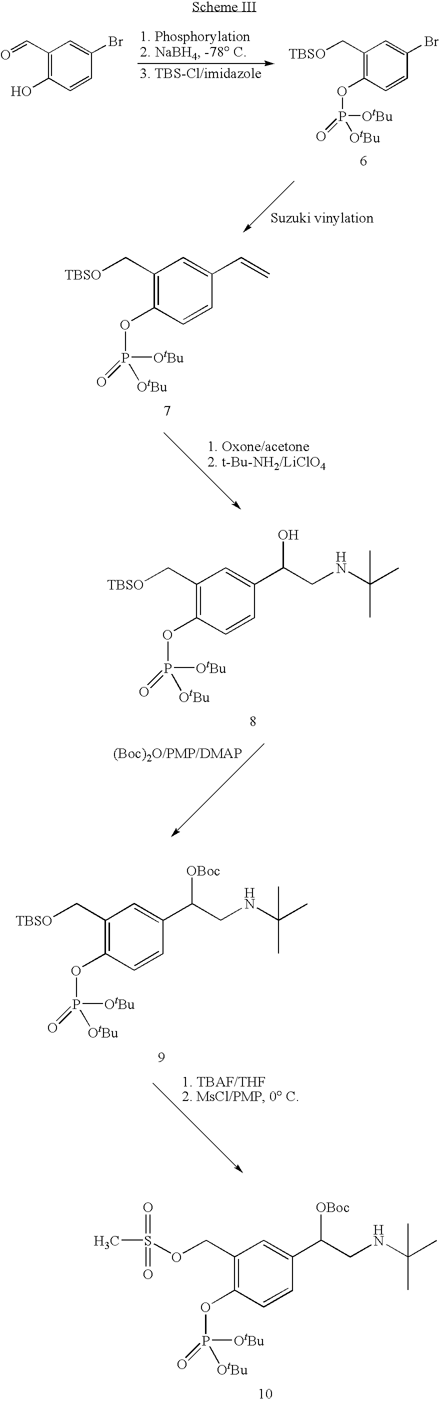

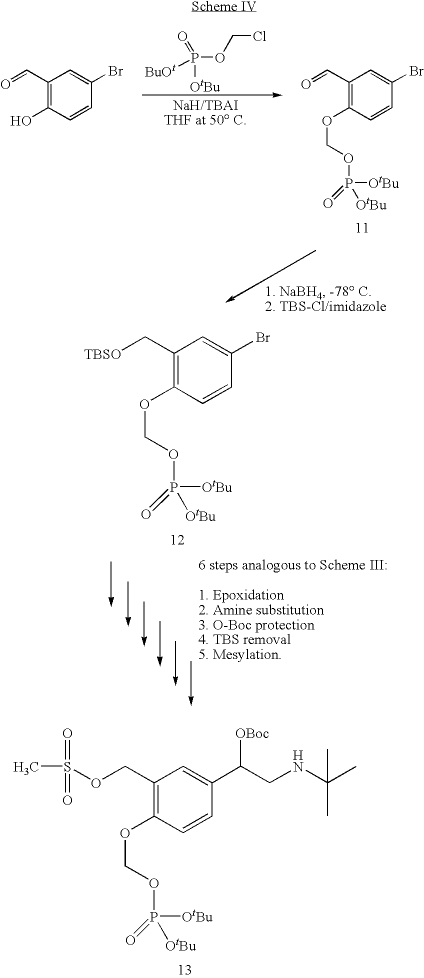

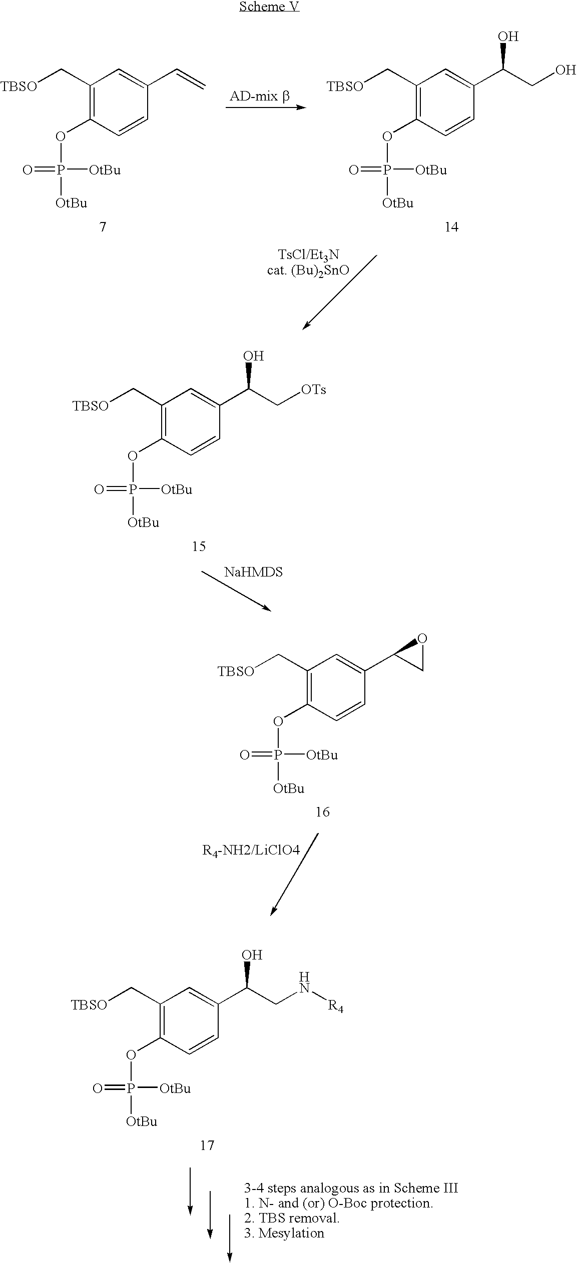

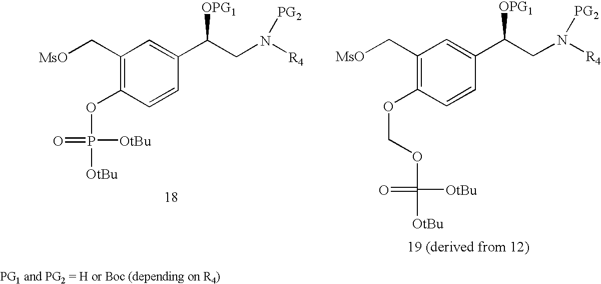

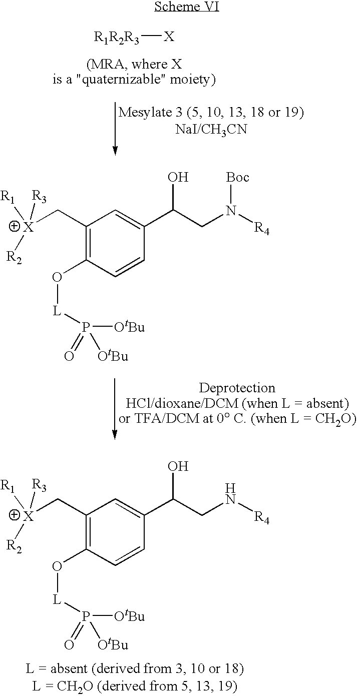

Synthesis of the phosphate-functionalized protected β-agonist derivative is shown in Schemes I-V.

Commercially available racemic salmeterol xinafoate (or prepared according to Rong and Ruoho, 1999) is protected with t-butoxycarbonyl group (Boc), followed by selective oxidation of the primary, benzylic alcohol to an aldehyde with activated MnO₂, yielding compound 1 (Example 3). In this manner the primary alcohol is disguised as an aldehyde and therefore the acidity of the phenolic moiety is increased, helping the selectivity of the subsequent phosphorylation. As a consequence the reaction with a slight excess of phosphobromidate (prepared as described in Example 1) proceeds cleanly, yielding the phosphate 2 in good yield and purity (Example 4). The reduction of the aldehyde moiety with sodium borohydride is carried out at low temperature (−78° C. to 0° C.) to produce the diol, which is selectively sulfonylated at about 0° C. using methanesulfonyl chloride (MsCl) in the presence of 1,2,2,6,6-pentamethylpiperidine (PMP) to give the primary mesylate 3 (Example 6). Thus an activated intermediate (Scheme I) is used in the alkylations linking the MRA molecule and a β-agonist into a mutual prodrug as depicted in Scheme VI.

Alternatively, the phosphono-oxymethyl derivative of salmeterol can be prepared as described in Scheme II. The phenolic moiety in compound 1 is alkylated at about 50° C. with di-tert-butyl chloromethyl phosphate (Krise et al., 1999) using sodium hydride as a base and tetrabutylammonium iodide as an auxiliary, yielding the derivative 4. The borohydride reduction of aldehyde, followed by the selective mesylation of the primary hydroxyl group (analogously as described in the preceding paragraph) gives the activated mesylate 5.

In the preparation of an albuterol derivative, the steric bulk around the aminoalcohol moiety (R₄=t-butyl) requires the indirect synthetic approach illustrated in Scheme III.

5-Bromosalicylaldehyde is phosphorylated and the aldehyde moiety reduced as described in the earlier paragraph, and the thus formed alcohol moiety is protected by treatment with tert-butyldimethylsilyl chloride in the presence of imidazole, yielding compound 6 (Examples 10-11). The presence of a bromine atom allows C—C bond formation in the following step. The trivinylboroxine-pyridine complex in the presence of catalytic amounts of tricyclohexylphosphine and palladium (II) acetate is used to introduce the vinyl substituent using the Suzuki method (Example 12). Thus formed compound 7 undergoes epoxidation by means of 2,2-dimethyldioxirane (DMDO) generated in situ in a mixture of oxone and acetone. The epoxide opening is accomplished by nucleophilic attack with tert-butylamine in the presence of lithium perchlorate as a Lewis acid ensuring regioselectivity resulting in a beta-aminoalcohol 8. Steric bulk imposed by the t-butyl moiety has impact on the subsequent acylation with di-t-butyl dicarbonate, which proceeds selectively on the secondary hydroxyl, rather than the secondary amine, yielding compound 9. The removal of silyl TBS protection is followed by low-temperature mesylation, which again, proceeds selectively on the primary, benzylic hydroxyl, producing mesylate 10 (with the hindered, secondary t-butylamine moiety untouched).

Alternatively, the phosphono-oxymethyl derivative of albuterol can be prepared as described in Scheme IV. The phenolic moiety in 5-bromosalicaldehyde is alkylated at about 50° C. with di-tert-butyl chloromethyl phosphate (Krise et al. 1999) using sodium hydride as a base and tetrabutylammonium iodide as an auxiliary, yielding the phosphorylated aldehyde 11. Subsequent reduction and silylation of the formed alcohol can lead to 12, which can then be transformed, analogously as described in Scheme III, into the mesylate 13.

If desired, the optically pure version of a salmeterol derivative can be obtained according to Schemes I and II, using a single, desired enantiomer prepared as described in literature (e.g. Hett et al. 1994).

An example of the alternative process towards the optically pure, phosphorylated β-agonist with an alternate side chain is illustrated in Scheme V. The vinyl compound 7 was asymmetrically dihydroxylated using AD-mix-beta, producing diol 14. The selective tosylation proceeds on the primary hydroxyl, which is ensured by the presence of a catalytic amount of dibutyltin oxide, thus forming intermediate 15. The chiral epoxide 16 is obtained by brief and low-temperature treatment with sodium hexamethyldisilazide as a base. The opening of the epoxide with the amine of choice (bearing the R₄ moiety) can lead to aminoalcohol 17, which can be later transformed through manipulation of protective groups and final mesylation into an activated, chiral intermediate 18. If the whole synthetic sequence described above is applied to bromocompound 12 as a substrate, the final result can be the mesylate analog 19.

Scheme VI illustrates the convergent assembly of the mutual prodrugs of an MRA and a β-agonist. The selected MRA's (prepared according to literature procedures) are alkylated with the benzylic mesylate of the protected, phosphorylated β-agonist derivatives (3, 5, 10, 13, 18 or 19) in the presence of a stoichiometric amount of sodium iodide in a polar, aprotic solvent like acetonitrile. In the final step, the intermediate quaternary ammonium salts are deprotected by mild acidolysis, either by brief (up to 1 hour) treatment with 4N HCl in dioxane or low-temperature treatment with TFA in dichloromethane at about 0° C., yielding the target mutual prodrugs of invention.

### II. Enzymatic Activation of Monophosphates as Mutual MRA-β-Agonist Prodrugs

Monophosphates described in the present invention (mutual prodrugs of MRAs and β-agonists) are designed to release both drugs in a multistep bioactivation process. First, alkaline phosphatase present in lungs (in the case of topical delivery) efficiently dephosphorylates the mutual prodrug triggering a cascade of chemical breakdown/hydrolysis that can be combined with subsequent enzymatic hydrolysis in the case of a double mutual prodrug (when MRA is additionally masked as an ester prodrug). It can be assumed that phosphate cleavage is not a rate determining step, occurring faster relative to the subsequent processes. The number of steps required and their respective kinetics depend on the structure of the mutual prodrug undergoing bioactivation. For example, if the methylenoxy-linker to a monophosphate moiety is present then the subsequent elimination of formaldehyde occurs at physiologic pH. The thus formed phenolate intermediate is highly prone to spontaneous hydrolysis occurring at the benzylic position, which “restores” the saligenin moiety of a O-agonist. That step is likely rate-determining and it might be influenced by the steric and electronic nature of the “leaving group” R₁R₂R₃X. The departing moiety R₁R₂R₃X is either a MRA itself, or its ester precursor, that in the final step of enzymatic cleavage by the nonspecific lung esterases delivers the MRA at the desired site of its action.

The bioactivation described above is depicted on Scheme VII and the examples of such transformation are described in Examples 85 and 86 (in vitro and in vivo, respectively).

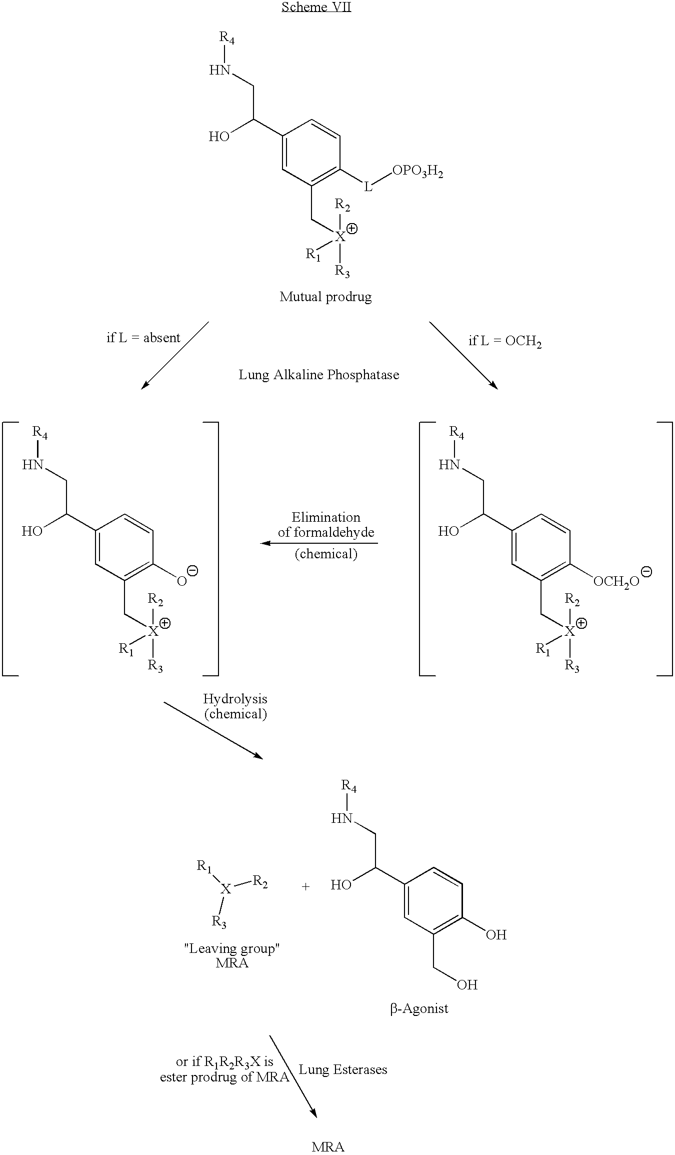

### III. Aerosol Delivery Devices

The use of the monophosphates as mutual MRA-β-agonist prodrugs suitably formulated for liquid nebulization, or alternatively as a dry powder provides sufficient amount of the mutual prodrug to the lungs to achieve a local therapeutic effect through the release of both bioactive components locally. Monophosphate mutual prodrugs of the invention are suitable for aerosolization using jet, electronic, or ultrasonic nebulizers. They are also appropriate for delivery by dry powder or metered dose inhaler. Their solid form has long-term stability permitting the drug substance to be stored at room temperature.

The aerosol formulation comprises a concentrated solution of about 1-10 mg/mL of pure monophosphate as a mutual MRA-β-agonist prodrug or its pharmaceutically acceptable salt, dissolved in aqueous or aqueous-ethanolic solution having a pH between about 4.0 and about 7.5. Preferred pharmaceutically acceptable salts are inorganic acid salts including hydrochloride, hydrobromide, sulfate or phosphate salts as they may cause less pulmonary irritation. The therapeutic amount of the mutual prodrug is delivered to the lung endobronchial space by nebulization of a liquid aerosol or dry powder having an average mass median diameter between about 1 to about 5μ. A liquid formulation may require separation of a mutual prodrug salt from the appropriate diluent requiring reconstitution prior to administration because the long-term stability of the monophosphate mutual prodrugs in aqueous solutions may not provide a commercially acceptable shelf life.

An indivisible part of this invention is a device able to generate aerosol from the formulation of the invention into aerosol particles predominantly in the 1-5μ size range. Predominantly, in this application, means that at least about 70% but preferably more than about 90% of all generated aerosol particles are within the 1-5μ size range. Typical devices include jet nebulizers, ultrasonic nebulizers, vibrating porous plate nebulizers, and energized dry powder inhalers.

A jet nebulizer utilizes air pressure to break a liquid solution into aerosol droplets. An ultrasonic nebulizer works by a piezoelectric crystal that shears a liquid into small aerosol droplets. A pressurized nebulization system forces solution under pressure through small pores to generate aerosol droplets. A vibrating porous plate device utilizes rapid vibration to shear a stream of liquid into appropriate droplet sizes. However, only some formulations of monophosphate mutual prodrugs can be efficiently nebulized, as the devices are sensitive to the physical and chemical properties of the formulation. Typically, the formulations which can be nebulized must contain small amounts of the monophosphate mutual prodrugs, which are delivered in small volumes (50-250 μL) of aerosol.

### IV. Utility

The compounds of the invention are useful (in humans) for treating pulmonary bronchoconstriction.

The amount of active ingredient that may be combined with the carrier materials to produce a single dosage form will vary depending upon the host treated and the particular mode of administration.

This small volume, high concentration formulation of monophosphate MRA-β-agonist prodrug can be delivered as an aerosol and at efficacious concentrations to the respiratory tract in patients suffering from mild to severe asthma, chronic bronchitis or chronic obstructive pulmonary disease (COPD). The solid dosage formulation is stable, readily manufactured and very cost effective. Furthermore, the formulation provides adequate shelf life for commercial distribution. The mutual prodrug masks the systemic side effects of MRAs, like dry mouth, pupil dilation or GI disturbances. The prodrug also masks the β-agonist activity minimizing a chance for cardiovascular side effects. Both drugs are released by enzymes present in lungs, specifically alkaline phosphatase, or in case of double mutual prodrug also involving esterases. Thereby the therapeutic amount of β-agonist and of a MRA are simultaneously released at the site of bronchoconstriction.

The foregoing may be better understood from the following examples, which are presented for the purposes of illustration and are not intended to limit the scope of the inventive concepts. The content of all references cited herein is incorporated by reference.

### Example 1

### Phosphorobromidic Acid Di-Tert-Butyl Ester

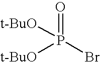

The title phosphorylating agent was prepared according to modified conditions compared to those described by Gajda and Zwierzak (1976). By lowering the temperature of the reaction to 15° C. and decreasing the reaction time to 2.5 hours the title compound obtained in our hands had better purity then when applying the literature conditions (25° C. for 4 hours). The title phosphobromidate is unstable and was immediately used for the phosphorylation reactions (see Examples 4 and 10).

Examples 2-6 illustrate the synthesis of the racemic phosphorylated derivative of salmeterol (see Scheme I).

### Example 2

### [2-Hydroxy-2-(4-hydroxy-3-hydroxymethyl-phenyl)-ethyl]-[6-(4-phenyl-butoxy)-hexyl-carbamic acid tent-butyl ester

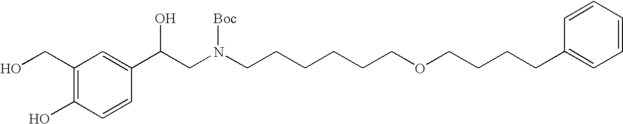

Commercially available salmeterol xinafoate (6.04 g, 10 mmol) and potassium carbonate (1.39 g, 10 mmol) were suspended with stirring in a 1,4-dioxane/water mixture (1:1, 80 mL). Then, di-t-butyl-dicarbonate (2.40 g, 11 mmol) dissolved in 1,4-dioxane (10 mL) was added dropwise while continuing stirring at room temperature. The TLC analysis after 30 minutes showed only traces of starting material. After 2 hours 1,4-dioxane was evaporated and the suspension formed was diluted with water and extracted twice with chloroform (125 mL total). Then, the organic layer was washed with saturated sodium bicarbonate, brine and dried over anhydrous magnesium sulfate. The crude material obtained after decantation and evaporation was purified by silica gel chromatography eluting with the ethyl acetate/hexane mixture (1:1). The title compound (4.61 g, 89%) was obtained as a glassy residue solidifying upon refrigeration. LCMS: 100%, MNa⁺538.3 (exact mass 515.3 calcd for C₃₀H₄₅NO₆). Anal. Calc: C, 69.87; H, 8.80; N, 2.72. Found: C, 69.69; H, 8.64; N. 2.68.

### Example 3

### [2-(3-Formyl-4-hydroxy-phenyl)-2-hydroxy-ethyl]-[6-(4-phenyl-butoxy)-hexyl]-carbamic acid tert-butyl ester

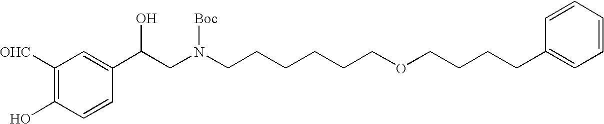

The N-Boc-salmeterol described in Example 2 (3.24 g, 6.28 mmol) was dissolved in chloroform (50 mL) and the activated manganese oxide (IV) (6.44 g, 85% w/w, 63 mmol) was added in portions with vigorous stirring. After 24 hours at room temperature the slurry was filtered through a pad of Celite, followed by concentration of the filtrate combined with the chloroform washes. The crude residue thus obtained was purified by silica gel chromatography using ethyl acetate/hexane mixture (1:5) yielding the title aldehyde 1 (2.45 g, 77%). LCMS: 96%, MNa⁺ 536.3 (exact mass 513.3 calcd for C₃₀H₄₃NO₆).

### Example 4

### {2-[4-(Di-tert-butoxy-phosphoryloxy)-3-formyl-phenyl]-2-hydroxy-ethyl}-[6-(4-phenyl-butoxy)-hexyl]-carbamic acid tert-butyl ester

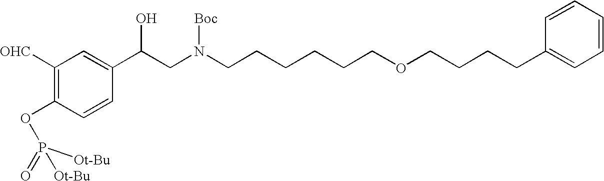

Aldehyde 1 (3.44 g, 6.69 mmol) was dissolved in anhydrous THF (10 mL), which was followed by adding DMAP (82 mg, 0.67 mmol) and DBU (1.11 mL, 7.4 mmol) with vigorous stirring under nitrogen. After cooling the reaction mixture to 0° C. the phosphobromidate described in Example 1 (2.19 g, 8 mmol) diluted with anhydrous THF (5 mL) was added dropwise over 15 minutes Stirring under nitrogen at 0° C. was continued for another 30 minutes, after which the TLC analysis showed the phosphorylation to be almost complete. After another 60 minutes the reaction mixture was concentrated, the residue was redissolved in ethyl acetate, washed 3 times with 10% citric acid, twice with 0.5N NaOH, brine and dried over anhydrous sodium sulfate. The organic phase was then filtered through a pad of basic alumina and the filtrate combined with ethyl acetate washes was concentrated in vacuo. The crude product was purified by silica gel chromatography using 30% ethyl acetate/1% triethylamine in hexane, yielding the title compound 2 (3.42 g, 72%) as a glassy residue.

³¹PNMR (CDCl₃): −15.107 ppm. LCMS: 100%, MNa⁺ 728.0 (exact mass 705.4 calcd for C₃₈H₆₀NO₉P). Anal. Calc: C, 64.66; H, 8.57; N, 1.98. Found: C, 64.09; H, 8.54; N, 2.02.

### Example 5

### {2-[4-Di-tert-butoxy-phosphoryloxy)-3-hydroxymethyl-phenyl]-2-hydroxy-ethyl}-[6-(4-phenyl-butoxy)-hexyl]-carbamic acid tert-butyl ester

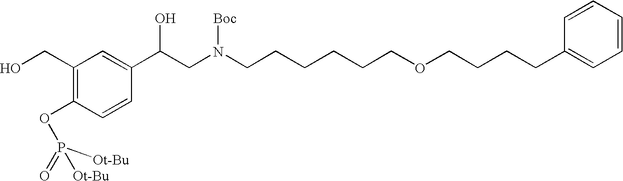

The phosphorylated aldehyde 2 (2.68, 3.8 mmol) was dissolved in anhydrous THF (10 mL) and the mixture was cooled to −78° C. Then, solid sodium borohydride (0.432 g, 11.4 mmol) was added in portions over 5 minutes with vigorous stirring under nitrogen, which was followed by adding methanol (1 mL). The reaction mixture was stirred allowing the temperature of the bath to increase to 0° C. over 4 hours (during which the TLC analysis showed consumption of the starting material). The reaction mixture was diluted with dichloromethane (50 mL), followed by careful quenching by adding 10% citric acid (20 mL) with vigorous stirring. The organic phase was separated, aqueous layer extracted with another portion of DCM and combined extracts were washed twice with saturated bicarbonate, brine, dried over anhydrous sodium sulfate, decanted and evaporated. The crude product was purified by chromatography using 40% ethyl acetate/1% triethylamine in hexane, yielding the title diol (2.01 g, 75%) as a colorless glassy residue.

¹H NMR (CDCl₃) selected signals: 7.17-7.41 (m, 8H), 4.92 (m, 1H), 4.62 (bs, 2H), 3.39 (q, 2H), 2.64 (t 21-1), 1.62 (m, 4H), 1.54 (s, 9H), 1.52 (s, 9H). 1.49 (s, 9H), 1.115-1.49 (m, 8H). ³¹PNMR (CDCl₃): −13.060 ppm. LCMS: 99%, MNa⁺ 730.0 (exact mass 707.4 calcd for C₃₈H₆₂NO₉P). Anal. Calc: C, 64.48; H, 8.83; N, 1.98. Found: C, 64.70; H, 8.84; N, 1.90.

### Example 6

### Methanesulfonic acid 5-(2-{tert-butoxycarbonyl-[6-(4-phenyl-butoxy)-hexyl]-amino}-1-hydroxy-ethyl)-2-(di-tert-butoxy-phosphoryloxy)-benzyl ester

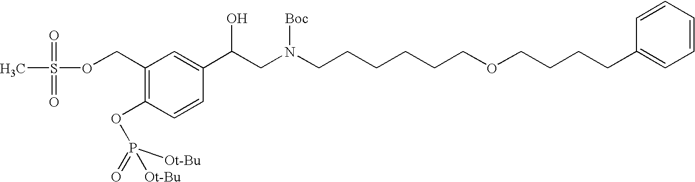

Compound 3 was synthesized by treating the diol described in Example 5 dissolved in anhydrous dichloromethane at 0° C. with 1.1 equivalents of methanesulfonyl chloride in the presence of 2 equiv. of 2,2,6,6-pentamethyl-piperidine (PMP). TLC monitoring showed the disappearance of the starting material after 15-30 minutes. After 1 hour the reaction mixture was concentrated in vacuo, redissolved in ethyl acetate, washed with 10% citric acid solution, saturated bicarbonate solution, brine, dried over anhydrous magnesium sulfate, decanted and evaporated. Thus obtained mesylate 3 was directly used for the quaternization (alkylation) of the MRA molecules (see Scheme VI).

Examples 7-9 illustrate the synthesis of the phosphonooxy-methylene derivative of salmeterol.

### Example 7

### {2-[4-(Di-tert-butoxy-phosphoryloxymethoxy)-3-formyl-phenyl]-2-hydroxy-ethyl}-[6-(4-phenyl-butoxy)-hexyl]-carbamic acid tert-butyl ester

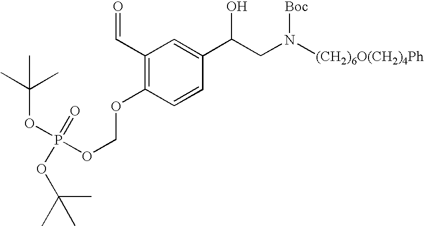

Salmeterol derivative 1 was alkylated with (t-BuO)₂P═O(OCH₂Cl) (1.2 equivalent added in portions—judges by TLC) according to a procedure analogous to the publication by Krise et al. (1999). Sodium hydride was used as a base (1 equivalent) and TBAI as a catalyst (0.2 equiv.) and the reaction was carried out in anhydrous THF with gentle heating (50° C.). Overall reaction time to consume the starting material was 18 hours, after which the mixture was cooled to room temperature and quenched with 10% (w/v) aqueous citric acid followed by THF removal via rotary evaporatoration. The resulting mixture was extracted with diethyl ether (twice) and the organic extracts were combined, and washed with: 0.5 M NaOH (3 times), 10% (w/v) aqueous citric acid, deionized water and brine, dried over anhydrous sodium sulfate and concentrated to yield crude 98% of brown, oily residue. That material was purified by silica gel chromatography, using the gradient (hexane/ethyl acetate—with both solvents buffered with 1% triethyl amine) to yield 70% of a clear, viscous oil. LC-MS MNa⁺=758 observed; HPLC with UV detector at 272 nm: 95 area %; ³¹P NMR in DMSO-d6 showed 2 peaks, consistent with product (−10.892 ppm, and mono des-t-butyl product (−11.529 ppm) with ratio of peak areas=96%.

### Example 8

### {2-[4-(Di-tert-butoxy-phosphoryloxymethoxy)-3-hydroxymethyl-phenyl]-2-hydroxy-ethyl}-[6-(4-phenyl-butoxy)-hexyl]-carbamic acid tert-butyl ester

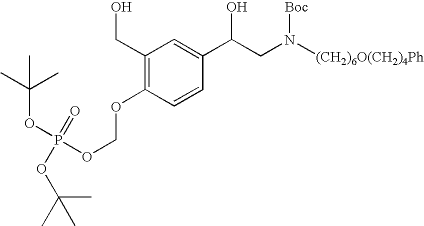

Aldehyde 4 was reduced analogously as described in Example 5, yielding the title compound in 92% yield of a slightly yellowish, viscous oil. LC-MS: MNa+=760 observed: HPLC at 272 nm: 96%. ³¹P NMR in DMSO-d6: −11.104 ppm.

### Example 9

### Methanesulfonic acid 5-(2-{tert-butoxycarbonyl-[6-(4-phenyl-butoxy)-hexyl]-amino}-1-hydroxy-ethyl)-2-(di-tert-butoxy-phosphoryloxymethoxy)-benzyl ester

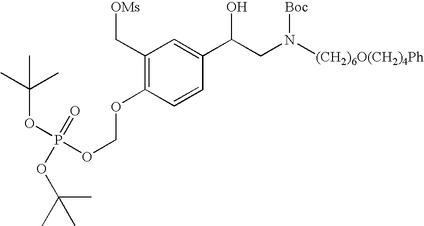

The diol described in Example 8 was selectively mesylated according to the procedure described in Example 6, yielding the mesylate 5 in high yield, which was used directly for quaternization reactions.

Examples 10-17 illustrate the synthesis of the racemic phosphorylated derivative of albuterol (see Scheme III).

### Example 10

### Phosphoric acid 4-bromo-2-formyl-phenyl ester di-tert-butyl ester

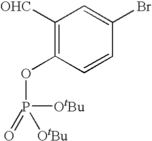

5-Bromosalicylaldehyde (8.04 g, 40 mmol) was phosphorylated analogously as described in Example 4, using DBU (6.58 mL, 44 mmol) and DMAP (0.489 g, 4 mmol) dissolved in anhydrous THF (50 mL) and cooled to 0° C. The phosphorylating agent was prepared as described in Example 1 (23.2 g, 85 mmol) and diluted with anhydrous THF (20 mL). The crude product was purified by chromatography (9% ethyl acetate+1% triethylamine in hexane) yielding analytically pure title aldehyde 6 as a yellowish solid (11.51 g, 73%).

¹HNMR (CDCl₃): 10.35 (s, 1H), 7.99 (d, 1H, J=2.4 Hz), 7.67 (dd, 1H, J=8.8 Hz, 2.4 Hz), 7.41 (d, 1H, J=8.8 Hz), 1.51 (s, 18H). ³¹PNMR (CDCl₃): −15.239 ppm. LCMS: 99%, MNa⁺415 (exact mass 392.04 calcd for C₁₅H₂₂BrO₅P).

### Example 11

### Phosphoric acid 4-bromo-2-(tert-butyl-dimethyl-silanyloxymethyl)-phenyl ester di-tert-butyl ester

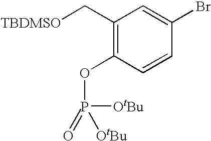

The aldehyde described in Example 10 was reduced to an alcohol analogously as described in Example 5. The crude material solidified upon repeated evaporation with hexane and was sufficiently pure to continue the synthesis. The intermediate alcohol was converted to compound 6 by treatment with slight excess of tert-butyldimethylsilyl chloride in DMF in the presence of excess (5 equivalents) of imidazole. After the overnight reaction at room temperature the mixture was diluted with diethyl ether, washed extensively with 10% citric acid and brine, and the organic phase was then dried with anhydrous magnesium sulfate, decanted and evaporated. The crude material was purified by chromatography using 10% ethyl acetate+1% triethylamine in hexane.

### Example 12

### Phosphoric acid di-tert-butyl ester 2-(tert-butyl-dimethyl-silanyloxymethyl)-4-vinyl-phenyl ester

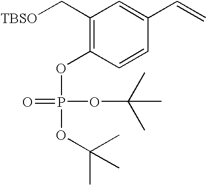

A two-neck, round bottomed flask, equipped with a reflux condenser was charged with a solution of compound 6 in a mixture of toluene (8 mL/mmol) and ethanol (1 mL/mmol) followed by adding a degassed 20% solution of potassium carbonate (8 mL/mmol). The biphasic mixture was vigorously stirred for 1 hour while a stream of argon was passed through the flask. To this mixture, trivinylboroxine-pyridine complex (1.5 equivalents) was added, followed by tricyclohexylphosphine (0.1 equivalent). The reaction mixture was purged with argon once again for 30 minutes, then palladium (II) acetate (0.1 equivalents) was added, followed by vigorous stirring and heating under reflux under the positive pressure of argon for 4 hours. After that time, TLC analysis (chloroform/methanol 8:1) showed the complete consumption of starting material. The reaction mixture was diluted with ethyl acetate (3 times the original volume) and the organic phase was washed with water (3 times), 10% citric acid solution (twice) and brine and was dried over anhydrous MgSO₄. After filtration and evaporation of the solvent, the residue was purified by silica gel chromatography (ethyl acetate/hexanes 1:20 with 5% of triethylamine), yielding 80% of the desired olefin 7 as a viscous oil.

¹H NMR (CDCl₃): 7.52 (s, 1H), 7.27 (d, 1H), 7.19 (d, 1H), 6.67 (dd, 1H), 5.66 (d, 1H), 5.17 (d, 1H). 4.71 (s, 2H), 1.48 (s, 18H), 0.95 (s, 9H), 0.10 (s, 6H). ³¹P NMR (CDCl₃): −14.18 ppm. LCMS: 95%, MNa+ 479 (exact mass 456.3 calcd for C₂₃H₄₁O₅PSi).

### Example 13

### Phosphoric acid di-tert-butyl ester 2-(tert-butyl-dimethyl-silanyloxymethyl)-4-oxiranyl-phenyl ester

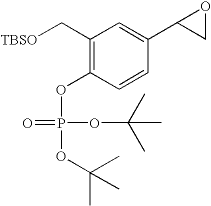

Oxone® (8 g, 13.1 mmol) was slowly added to a stirring solution of compound 7 (1.2 g, 2.63 mmol) in a CH₂Cl₂/satd NaHCO₃ mixture (20 mL, 3:5) and acetone (10 mL) at 0° C. The pH of the mixture was adjusted to >7.5 with satd NaHCO₃ as needed. After stirring for 30 minutes at 0° C., then 90 minutes at room temperature the resulting suspension was extracted with CH₂Cl₂ (3×15 mL), dried over Na₂SO₄ and concentrated to give crude epoxide (1.3 g) as light yellow oil. Chromatography (3:1 hexanes/ethyl acetate, 0.5% Et₃N) afforded the title epoxide (0.804 g, 65%) as clear oil: ¹HNMR (400 MHz, DMSO-D6) δ 7.36 (s, 1H), 7.23 (m, 2H), 4.74 (s, 2H), 3.92 (dd, 1H, J=2.6, 4.1), 3.11 (dd, 1H, J=4.1, 5.3), 2.77 (dd, 1H, J=2.6, 5.3), 1.43 (s, 18H), 0.90 (s, 9H), 0.08 (s, 6H).

### Example 14

### Phosphoric acid di-tert-butyl ester 4-(2-tert-butylamino-1-hydroxy-ethyl)-2-(tert-butyl-dimethyl-silanyloxymethyl)-phenyl ester

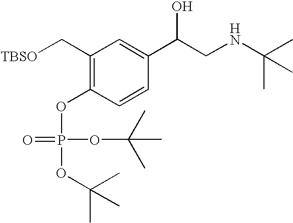

Solid LiClO₄ (180 mg, 1.7 mmol) was added to a stirring solution of epoxide described in Example 13 (4 g, 8.5 mmol) in tert-butylamine (9 mL, 84 mmol) while stirring at room temperature. The resulting mixture was stirred for 48 hours, then diluted with ethyl acetate (20 mL). The organic layer was washed with water, brine, dried over Na₂SO₄ and concentrated to give crude aminoalcohol (5.3 g) as yellow oil. Chromatography (9:1, CH₂Cl₂/MeOH, 0.5% Et₃N) afforded the title compound 8 (4.2 g, 91%) as light yellow oil.

¹H NMR (400 MHz, DMSO-D6) δ 7.45 (s, 1H), 7.23 (dd, 1H, J=2.1, 8.4), 7.18 (d, 1H, J=9.0), 4.75 (s, 2H), 4.49 (t, 1H, J=6.2), 3.17 (s, 1H), 2.58 (d, 2H, J=6.3), 1.42 (m. 181-1). 1.01 (d, 9H, J=14.4), 0.92 (s, 9H), 0.06 (s, 6H); ES/MS, calcd for C₂₇H₅₃NO₆PSi 546.34, found M/Z=546.4 (M+H).

### Example 15

### Carbonic acid tert-butyl ester 2-tert-butylamino-1-[3-(tert-butyl-dimethyl-silanyloxymethyl)-4-(di-tert-butoxy-phosphoryloxy)-phenyl]-ethyl ester

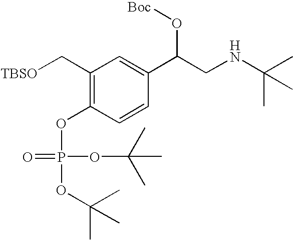

Solid (Boc)₂O (1.04 g, 4.79 mmol) was added to a stirred solution of 8 (1.74 g, 3.19 mmol), PMP (1.7 mL, 9.6 mmol), and DMAP (39 mg, 0.319 mmol) in anhydrous CH₃CN (30 mL) at 0° C. After 90 minutes the resulting mixture was quenched with saturated NaHCO₃ (40 mL) and extracted with ethyl acetate (3×30 mL). The combined organic layers were washed with brine, dried over Na₂SO₄, and concentrated to give crude carbonate (2.93 g) as white solid. Chromatography (1:3, hexanes/ethyl acetate, 0.5% Et₃N) afforded the title compound 9 (0.946 g, 46%) as clear oil.

¹H NMR (400 MHz, DMSO-D6) δ 7.43 (s, 1H), 7.23 (m, 2H), 5.38 (dd, 1H, J=5.0, 7.7), 4.75 (s, 2H), 2.79 (m, 2H), 1.43 (s, 18H), 1.36 (s, 9H), 0.96 (s, 9H), 0.92 (s, 9H), 0.07 (m, 6H); ES/MS, calcd for C₃₂H₆₁NO₈PSi 646.39, found m/z=646.5 (M±H).

### Example 16

### Carbonic acid tert-butyl ester 2-tert-butylamino-1-[4-(di-tert-butoxy-phosphoryloxy)-3-hydroxymethyl-phenyl]-ethyl ester

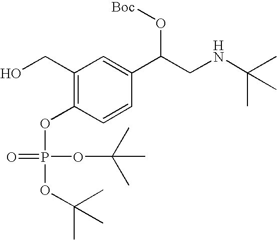

A 1.0M solution of TBAF in THF (1.4 mL, 1.4 mmol) was added to a stirred solution of compound 9 (0.9 g, 1.4 mmol) in anhydrous THF (14 mL) at room temperature. The resulting suspension was stirred for 1 hour, then quenched with satd NaHCO₃ (20 mL) and the aqueous layer was extracted with ethyl acetate (3×20 mL). The combined organic layers were washed with brine, dried over Na₂SO₄, and concentrated to give crude alcohol (1.01 g) as light yellow oil. Chromatography (1:3, hexanes/ethyl acetate, 0.5% Et₃N) afforded pure title compound (0.61 g, 82%) as a clear oil.

¹H NMR (400 MHz, DMSO-D6) δ 7.45 (s, 11-1). 7.21 (m, 2H), 5.40 (dd, 1H, J=4.8, 8.0), 5.22 (t, 1H, J=5.6), 4.56 (d, 2H, J=5.5). 2.79 (ddd, 2H, J=6.5, 12.3, 17.1), 1.43 (m, 18H), 1.37 (s, 9H), 0.98 (s, 9H); ES/MS, calcd for C₂₆H₄₇NO₈P 532.30, found m/z=532.4 (M+H).

### Example 17

### Methanesulfonic acid 5-[2-(tert-butoxycarbonyl-tert-butyl-amino)-1-hydroxy-ethyl]-2-(di-tert-butoxy-phosphoryloxy)-benzyl ester

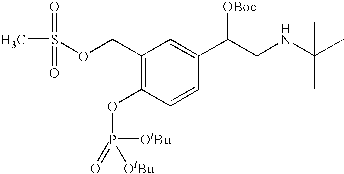

A solution of methanesulphonyl chloride (105 μL, 1.36 mmol) in CH₂Cl₂ (0.5 mL) was added dropwise to a stirred solution of compound described in Example 16 (0.6 g, 1.13 mmol) and PMP (817 μL, 4.52 mmol) in CH₂Cl₂ (12 mL) at 0° C. The reaction mixture was stirred for 30 minutes then quenched with satd NaHCO₃ (20 mL). The organic layer was separated, dried over Na₂SO₄, and concentrated to give crude mesylate (0.98 g) as light yellow oil. Chromatography (1:3, hexanes/ethyl acetate, 0.5% Et₃N) afforded the title mesylate 10 (0.56 g, 76%) as a clear oil. ES/MS, calcd for C₂₇H₄₉NO₁₀PS 610.28, found m/z=610.4 (M+H).

Examples 18-25 illustrate the synthesis of phosphonooxy-methylene derivative of racemic albuterol (salbutamol).

### Example 18

### Phosphoric acid 4-bromo-2-formyl-phenoxymethyl ester di-tert-butyl ester

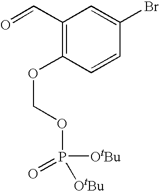

The title compound 11 can be synthesized analogously as described in Example 7, using the 5-bromosalicaldehyde as a starting material.

### Example 19

### Phosphoric acid 4-bromo-2-(tert-butyl-dimethyl-silanyloxymethyl)-phenoxymethyl ester di-tert-butyl ester

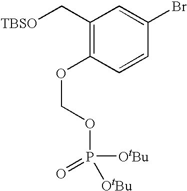

The title compound 12 can be synthesized analogously as described in Example 11, using the aldehyde 11 as a starting material.

### Example 20

### Phosphoric acid di-tert-butyl ester 2-(tert-butyl-dimethyl-silanyloxymethyl)-4-vinyl-phenoxymethyl ester

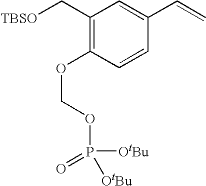

The title compound can be synthesized by the Suzuki vinylation described in Example 12, using the bromocompound 12 as a starting material.

### Example 21

### Phosphoric acid di-tert-butyl ester 2-(tert-butyl-dimethyl-silanyloxymethyl)-4-oxiranyl-phenoxymethyl ester

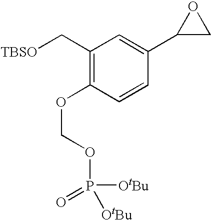

The title compound can be synthesized through epoxidation described in Example 13, using the compound described in Example 20 as a starting material.

### Example 22

### Phosphoric acid di-tert-butyl ester 4-(2-tert-butylamino-1-hydroxy-ethyl)-2-(tert-butyl-dimethyl-silanyloxymethyl)-phenoxymethyl ester

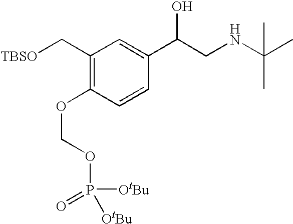

The aminolysis with t-butylamine (as described in Example 14) can be used to synthesize the compound depicted above using compound from Example 21 as a substrate.

### Example 23

### Carbonic acid tert-butyl ester 2-tert-butylamino-1-[3-(tert-butyl-dimethyl-silanyloxymethyl)-4-(di-tert-butoxy-phosphoryloxymethoxy)-phenyl]-ethyl ester

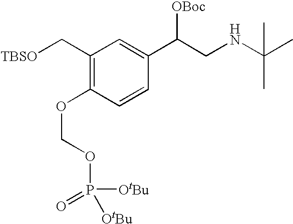

The O-acylation (protection) of the aminoalcohol described in Example 22 can be accomplished according to the procedure described in Example 15.

### Example 24

### Carbonic acid tert-butyl ester 2-tert-butylamino-1-[4-(di-tert-butoxy-phosphoryloxymethoxy)-3-hydroxymethyl-phenyl]-ethyl ester

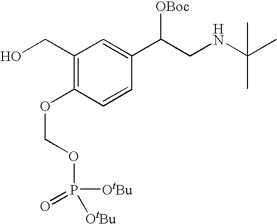

The TBS-removal from compound described in previous Example can be achieved analogously as described in Example 16.

### Example 25

### Methanesulfonic acid 5-(1-tert-butoxycarbonyloxy-2-tert-butylamino-ethyl)-2-(di-tert-butoxy-phosphoryloxymethoxy)-benzyl ester

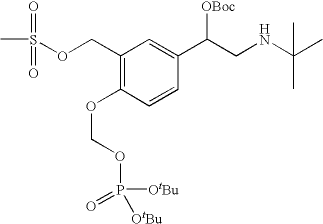

Title compound 13 can be synthesized according to procedure described in Example 17, using the aminoalcohol from Example 24 as a substrate.

Examples 26-28 illustrate the synthesis of the asymmetric intermediate, that can be used to prepare optically pure β-agonist derivatives (see Scheme V).

### Example 26

### Phosphoric acid di-tert-butyl ester 2-(tert-butyl-dimethyl-silanyloxymethyl)-1-(1,2R-dihydroxy-ethyl)-phenyl ester

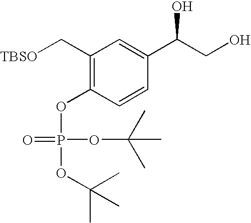

A solid AD-mix β reagent (300 mg) was added to a stirred solution of 7 (100 mg, 0.219 mmol) in t-BuOH (1 mL) and H₂O (1 mL) at 0° C. After stirring for 19 hours, solid Na₂SO₃ (300 mg) was added to quench and the resulting reaction mixture was allowed to warm up to room temperature and stirred for an additional 1 hour. After being diluted with water the reaction mixture was extracted with CH₂Cl₂ (3×15 mL). The combined organic layers were dried over Na₂SO₄ and concentrated to give crude diol (123 mg) as pale yellow oil. Chromatography (1:3, hexanes/ethyl acetate, 0.5% Et₃N) afforded title compound 14 (93 mg, 87%) as clear oil.

¹H NMR (400 MHz, DMSO-D6) δ 7.46 (d, 1H, J=8.4 Hz), 7.18 (m, 2H), 5.20 (brd, 2H, J=48.0 Hz), 4.53 (m, 3H), 3.41 (d, 21-1, J=6.7 Hz), 1.43 (s, 18H), 0.83 (s, 61-1), −0.06 (s, 6H); ES/MS calcd for C₂₃H₄₃NaO₇PSi 513.24, found m/z=513.3 (M+Na).

### Example 27

### Toluene-4-sulfonic acid 2-[3-(tert-butyl-dimethyl-silanyloxymethyl)-4-(di-tert-butoxy-phosphoryloxy)phenyl]-2R-hydroxy-ethyl ester

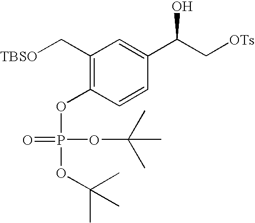

To a stirred solution of compound 14 (660 mg, 1.35 mmol) in CH₂Cl₂ (13 mL) dibutyltinoxide (0.7 mg, 0.0027 mmol), Et₃N (188 μL, 1.35 mmol), and TsCl (257 mg, 1.35 mmol) were added in the aforementioned order at room temperature. The reaction mixture was stirred for 90 minutes and then quenched with H₂O (20 mL). The aqueous layer was extracted with CH₂Cl₂ (3×15 mL). The combined organic layers were dried over Na₂SO₄ and concentrated to give crude monotosylate (1.19 g) as opaque semi solid. Chromatography (1:1, hexanes/ethyl acetate, 0.5% Et₃N) afforded pure 15 (700 mg, 81%) as clear oil.

¹H NMR (400 MHz, DMSO-D6) δ 7.67 (m, 2H), 7.43 (m. 2H), 7.36 (s, 1H), 7.18 (m, 2H). 5.80 (d, 1H, J=4.6 Hz), 4.76 (dd, 1H, J=5.3, 10.3 Hz), 4.71 (s, 2H), 3.95 (d, 2H, J=6.1 Hz), 2.40 (s, 3H), 1.43 (s, 18H), 0.89 (m, 9H), 0.05 (d, 6H, J=0.6 Hz); ES/MS calcd for C₃₀H₄₉NaO₉PSSi 667.25, found m/z=667.2 (M+Na).

### Example 28

### Phosphoric acid di-tert-butyl ester 2-(tert-butyl-dimethyl-silanyloxymethyl) —(S)-4-oxiranyl-phenyl ester

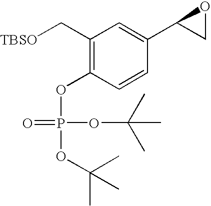

A 1.0M solution of NaHMDS in THF (1.3 mL, 1.30 mmol) was added dropwise to a stirred solution of 15 (420 mg, 0.651 mmol) in THF (7 mL) at 0° C. The resulting mixture was stirred for additional 10 minutes, quenched with satd NaHCO₃ (15 mL) and extracted with ethyl acetate (3×20 mL). The combined organic layers were washed with brine, dried over Na₂SO₄, and concentrated to give crude epoxide (293 mg) as pale yellow semi solid. Chromatography (3:1, hexanes/ethyl acetate, 0.5% Et₃N) afforded title compound 16 (250 mg, 81%) as clear oil.

¹H NMR (400 MHz, DMSO-D6) δ 7.36 (s, 1H), 7.23 (d, 2H, J=1.2 Hz), 4.74 (s, 2H), 3.93 (dd, 1H, J=2.6, 4.1 Hz), 3.11 (dd, 1H, J=4.1, 5.3 Hz), 2.78 (dd, 1H, J=2.6, 5.3 Hz), 1.41 (d, 18H, J=15.4 Hz), 0.90 (m, 9H), 0.06 (m, 6H).

Examples 29-84 illustrate the mutual prodrugs of MRAs and beta-agonists, prepared according to Scheme VI.

### Example 29

### 1-(5-{1-Hydroxy-2-[6-(4-phenyl-butoxy)-hexylamino]-ethyl}-2-phosphonooxy-benzyl)-4-{[(1-{4-hydroxy-1-[3,3,3-tris-(4-fluoro-phenyl)-propionyl]-pyrrolidine-(S)-2-carbonyl}-pyrrolidine-(R)-2-carbonyl)-amino]-methyl}-1-methyl-piperidinium (GS343071)

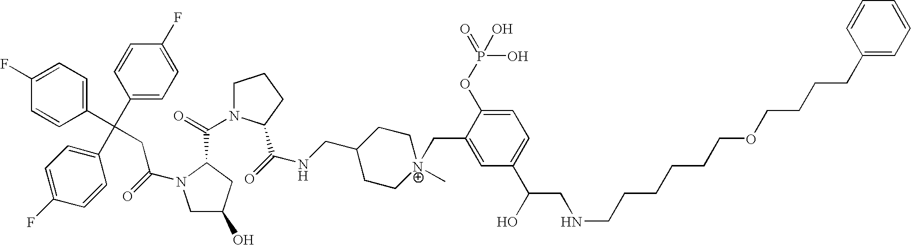

Quaternization step. A solution of 1-{4-hydroxy-1-[3,3,3-tris-(4-fluoro-phenyl)-propionyl]-pyrrolidine-(S)-2-carbonyl}-pyrrolidine-(R)-2-carboxylic acid-(1-methyl-piperidin-4-ylmethyl)-amide (100 mg, 0.148 mmol), prepared as described by Sagara et al. (2006), and the mesylate 3 (196 mg, 0.222 mmol) in acetonitrile (1 mL) was charged with sodium iodide (22 mg, 0.148 mmol) and stirring at room temperature was continued for 24 hours. The reaction mixture was concentrated, then redissolved in dichloromethane (10 mL) and water (10 mL) and stirred. After 5 minutes, layers were separated and the dichloromethane layer was washed with brine (10 mL), dried (Na₂SO₄) and concentrated to provide crude, protected piperidinium salt (271 mg) as yellow oil. Chromatography (1:0 to 1:1 gradient CH₂Cl₂/MeOH, Teledyne-Isco 14 gram —NH₂ column) afforded 91 mg (0.067 mmol) of the mono-t-butyl-protected phosphate product as yellowish oil.

ES/MS, calcd for C₇₆H₁₀₄F₃N₅O₁₂P 1367.74 m/z (M+1)⁺; observed, 1367.9 m/z.

Deprotection and final purification. Product obtained from the quaternization step (91 mg, 0.067 mmol) was dissolved in anhydrous DCM (2 mL) to which a solution of HCl (2 mL, 4N in 1,4-dioxane) was added dropwise and stirred at room temperature. After 1 hour, the reaction was concentrated then triturated with diethyl ether. The resulting suspension was filtered to provide crude piperidinium salt (101 mg) as a white solid. Reverse-phase chromatography (1:0 to 0:1 gradient H₂O/ACN with 1% AcOH, Teledyne Isco 4.3 gram C18 column) afforded the title mutual prodrug (60 mg, 0.052 mmol) as a white solid.

¹H NMR (400 MHz, CD₃OD) δ ppm 7.68 (m, 1H), 7.42 (m, 2H), 7.30 (m, 1H), 7.19 (m, 9H), 7.00 (m, 5H), 4.51 (m, 5H), 3.80 (m, 2H), 3.64 (m, 3H), 3.51 (m, 2H), 3.42 (m, 5H), 3.36 (m, 1H), 3.26 (m, 1H), 3.14 (m, 3H), 3.00 (dd, J=11.12, 10.33 Hz, 5H), 2.62 (s, 2H), 2.54 (m, 1H), 2.24 (m, 1H), 2.12 (m, 2H), 2.01 (m, 1H), 1.95 (s, 1H), 1.88 (m, 2H). 1.80 (m, 1H), 1.67 (s, 5H), 1.58 (m, 4H), 1.42 (ddd, J=4.04, 1.37, 0.67 Hz, 4H), 1.30 (m, 1H); ¹⁹F NMR (400 MHz, CD₃OD) δ ppm −118.42 (s, 1F), −119.13 (m, 1F), −118.74 (m, 1F), −118.97 (m, 1F); ³¹P NMR (400 MHz, CD₃OD) δ ppm 75.00 (s, 1P); ES/MS, calcd for C₆₃H₈₀F₃N₅O₁₀P 1154.56 m/z (M)⁻; observed, 1154.6 m/z. Anal. Calcd: C, 63.14; H. 6.88; N, 5.50. Found: C. 58.64, H. 6.90, N, 5.39.

### Example 30

### 1-[5-(2-tert-Butylamino-1-hydroxy-ethyl)-2-phosphonooxy-benzyl]-4-{[(1-{4-hydroxy-1-[3,3,3-tris-(4-fluoro-phenyl)-propionyl]-pyrrolidine-2-carbonyl}-pyrrolidine-2-carbonyl)-amino]-methyl}-1-methyl-piperidinium

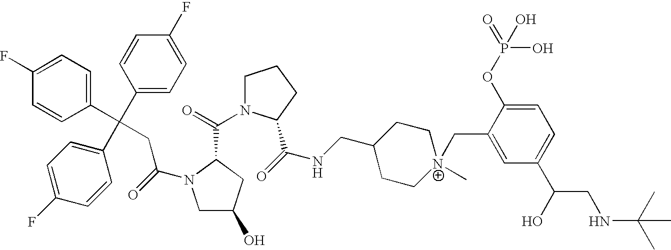

The title compound can be prepared through a two-step procedure described in Example 29 applied to 1-{4-hydroxy-1-[3,3,3-tris-(4-fluoro-phenyl)-propionyl]-pyrrolidine-(S)-2-carbonyl}-pyrrolidine-(R)-2-carboxylic acid-(1-methyl-piperidin-4-ylmethyl)-amide and mesylate 10 as starting materials.

### Example 31

### 1-(5-{1-Hydroxy-2-[6-(4-phenyl-butoxy)-hexylamino]-ethyl}-2-phosphonooxymethoxy-benzyl)-4-{[(1-{4-hydroxy-1-[3,3,3-tris-(4-fluoro-phenyl)-propionyl]-pyrrolidine-2-carbonyl}-pyrrolidine-2-carbonyl-amino]-methyl}-1-methyl-piperidinium

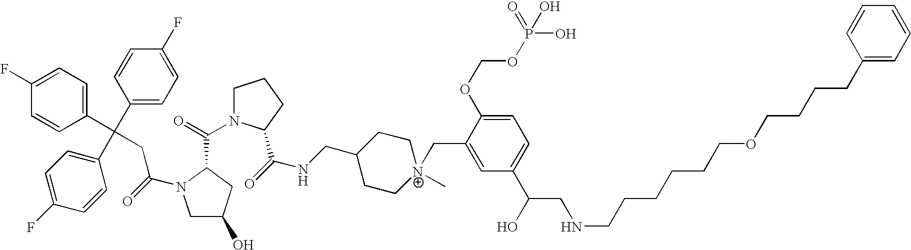

The title compound can be prepared through a two-step procedure described in Example 29 applied to 1-{4-hydroxy-1-[3,3,3-tris-(4-fluoro-phenyl)-propionyl]-pyrrolidine-(S)-2-carbonyl}-pyrrolidine-(R)-2-carboxylic acid-(1-methyl-piperidin-4-ylmethyl)-amide and mesylate 5 as starting materials.

### Example 32

### 1-[5-(2-tert-Butylamino-1-hydroxy-ethyl)-2-phosphonooxymethoxy-benzyl]-4-{[(1-{4-hydroxy-1-[3,3,3-tris-(4-fluoro-phenyl)-propionyl]-pyrrolidine-2-carbonyl}-pyrrolidine-2-carbonyl)-amino]-methyl}-1-methyl-piperidinium

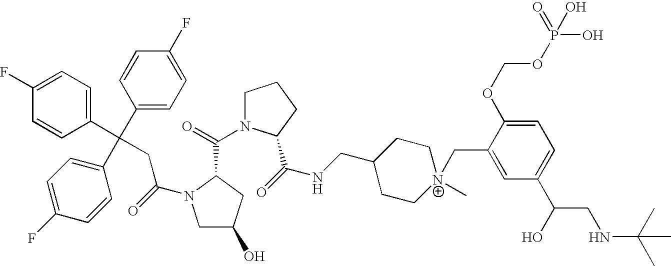

The title compound can be prepared through a two-step procedure described in Example 29 applied to 1-{4-hydroxy-1-[3,3,3-tris-(4-fluoro-phenyl)-propionyl]-pyrrolidine-(S)-2-carbonyl}-pyrrolidine-(R)-2-carboxylic acid-(1-methyl-piperidin-4-ylmethyl)-amide and mesylate 13 as starting materials.

### Example 33

### Monophosphate of (2-methylene-4-{1-hydroxy-2-[6-(4-phenyl-butoxy)-hexylamino]-ethyl}-phenyl)-3-[3-(2-Diethylamino-acetoxy)-2-phenyl-propionyloxy]-8-isopropyl-8-methyl-8-azonia-bicyclo[3.2.1]octane

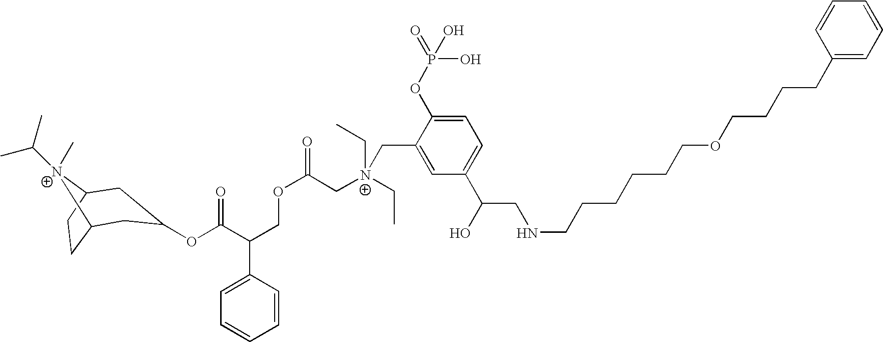

Esterification of ipratropium bromide. To a suspension of N,N-diethyl glycine sodium salt (459 mg, 3.00 mmol) and ipratropium bromide (861 mg, 2.00 mmol) in dichloromethane (6 mL) O-(7-Azabenzotriazol-1-yl)—N,N,N′,N′-tetramethyluronium hexafluorophosphate (HATU; 1141 mg, 3.00 mmol) was added and stirred vigorously. After 15 hours, the reaction was filtered and the solid was rinsed with dichloromethane. The filtrate and washes were combined and washed with sodium bicarbonate solution (twice) and brine, then dried (Na₂SO₄) and concentrated to provide crude ester (3-[3-(2-Diethylamino-acetoxy)-2-phenyl-propionyloxy]-8-isopropyl-8-methyl-8-azonia-bicyclo[3.2.1]octane—1.257 g) as a white semi-solid.

¹H NMR (400 MHz, DMSO-D6) δ ppm 7.50-7.28 (m, 1H), 5.03 (t, J=5.60 Hz, 1H), 5.75 (s, 1H). 4.59 (dd, J=10.91, 7.94 Hz, 1H), 4.41 (dd, J=10.93, 6.66 Hz, 1H), 4.18-3.74 (m, 1H), 3.23 (s, 1H), 2.58-2.38 (m, 2H), 2.33-2.13 (m, 1H), 2.13-1.94 (m, 1H), 1.81 (d, J=17.04 Hz, 1H), 1.66-1.48 (m, 1H). 1.23 (t, J=6.70 Hz, 1H). 0.89 (t, J=7.16 Hz, 1H); ES/MS, calcd for C₂₆H₄₁N₂O₄ 445.31 m/z (M)⁺; observed. 445.4 m/z.

The title compound can be prepared through a two-step procedure described in Example 29 applied to 3-[3-(2-Diethylamino-acetoxy)-2-phenyl-propionyloxy]-8-isopropyl-8-methyl-8-azonia-bicyclo[3.2.1]octane and mesylate 3 as starting materials.

### Example 34

### Monophosphate of 4-(2-tert-butylamino-1-hydroxy-ethyl)-2-methylene-phenyl]-3-[3-(2-Diethylamino-acetoxy)-2-phenyl-propionyloxy]-8-isopropyl-8-methyl-8-azonia-bicyclo[3.2.1]octane

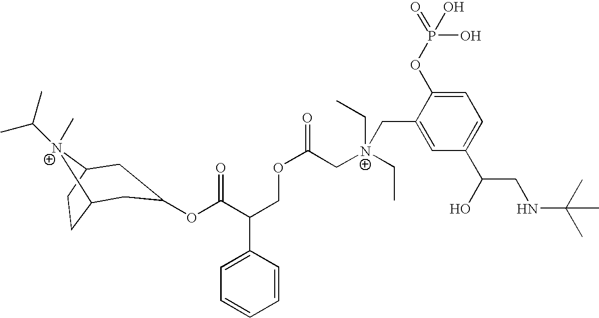

The title compound can be prepared through a two-step procedure described in Example 29 applied to 3-[3-(2-Diethylamino-acetoxy)-2-phenyl-propionyloxy]-8-isopropyl-8-methyl-8-azonia-bicyclo[3.2.1]octane and mesylate 10 as starting materials.

### Example 35

### Monophosphate of (2-methylene-4-{1-hydroxy-2-[6-(4-phenyl-butoxy)-hexylamino]-ethyl}-phenoxymethyl)-3-[3-(2-Diethylamino-acetoxy)-2-phenyl-propionyloxy]-8-isopropyl-8-methyl-8-azonia-bicyclo[3.2.1]octane

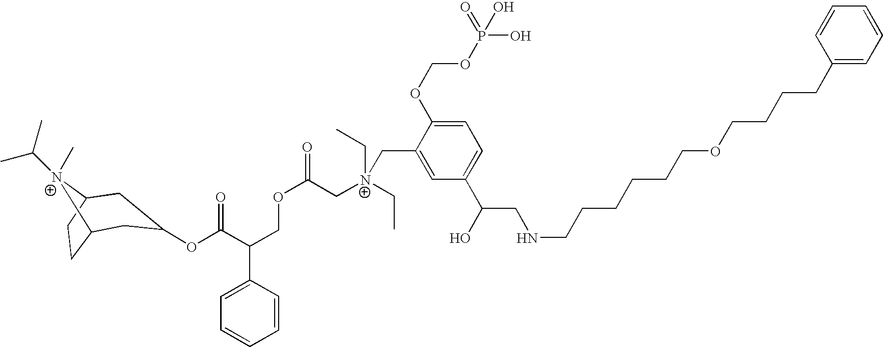

The title compound can be prepared through a two-step procedure described in Example 29 applied to 3-[3-(2-Diethylamino-acetoxy)-2-phenyl-propionyloxy]-8-isopropyl-8-methyl-8-azonia-bicyclo[3.2.1]octane and mesylate 5 as starting materials.

### Example 36

### Monophosphate of -[4-(2-tert-butylamino-1-hydroxy-ethyl)-2-methylene-phenoxymethyl]-3-[3-(2-Diethylamino-acetoxy)-2-phenyl-propionyloxy]-8-isopropyl-8-methyl-8-azonia-bicyclo[3.2.1]octane

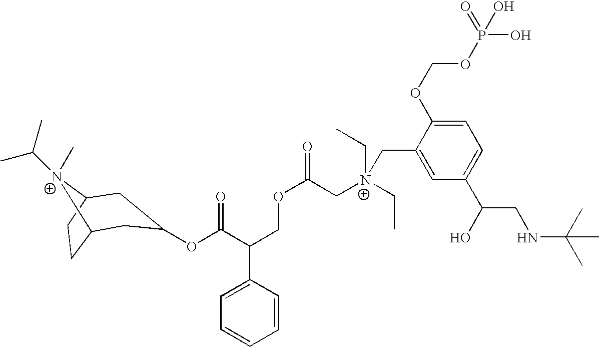

The title compound can be prepared through a two-step procedure described in Example 29 applied to 3-[3-(2-Diethylamino-acetoxy)-2-phenyl-propionyloxy]-8-isopropyl-8-methyl-8-azonia-bicyclo[3.2.1]octane and mesylate 13 as starting materials.

### Example 37

### 3-(1-Cyclohexyl-3,4-dihydro-1H-isoquinoline-2-carbonyloxy)-1-(5-{1-hydroxy-2-[6-(4-phenyl-butoxy)-hexylamino]ethyl}-2-phosphonooxy-benzyl)-1-azonia-bicyclo[2.2.2]octane

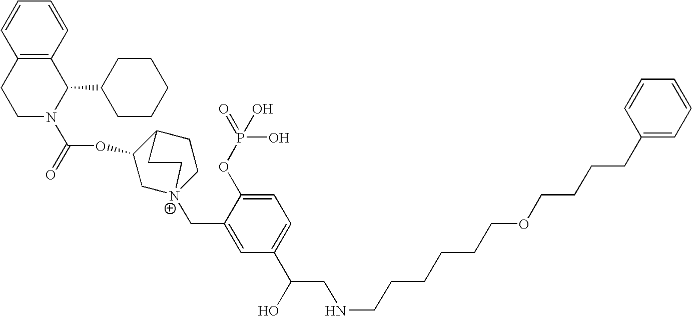

The title compound can be prepared through a two-step procedure described in Example 29 applied to Solifenacin (1-Cyclohexyl-3,4-dihydro-1H-isoquinoline-2-carboxylic acid 1-aza-bicyclo[2.2.2]oct-3-yl ester) and mesylate 3 as starting materials.

### Example 38

### 1-[5-(2-tert-Butylamino-1-hydroxy-ethyl)-2-phosphonooxy-benzyl]-3-(1-cyclohexyl-3,4-dihydro-1H-isoquinoline-2-carbonyloxy)-1-azonia-bicyclo[2.2.2]octane

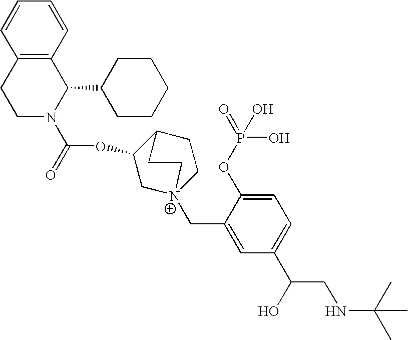

The title compound can be prepared through a two-step procedure described in Example 29 applied to Solifenacin (1-Cyclohexyl-3,4-dihydro-1H-isoquinoline-2-carboxylic acid 1-aza-bicyclo[2.2.2]oct-3-yl ester) and mesylate 10 as starting materials.

### Example 39

### 3-(1-Cyclohexyl-3,4-dihydro-1H-isoquinoline-2-carbonyloxy)-1-(5-{1-hydroxy-2-[6-(4-phenyl-butoxy)-hexylamino]-ethyl}-2-phosphonooxymethoxy-benzyl)-1-azonia-bicyclo[2.2.2]octane

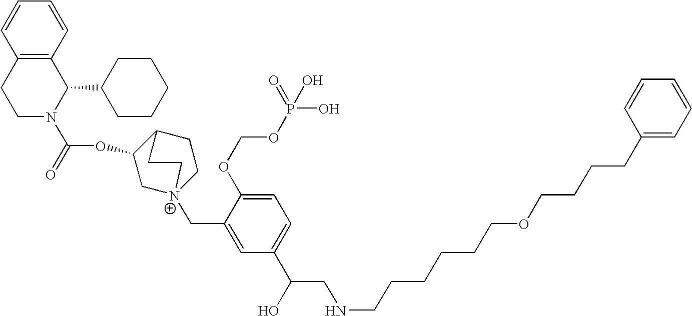

The title compound can be prepared through a two-step procedure described in Example 29 applied to Solifenacin (1-Cyclohexyl-3,4-dihydro-1H-isoquinoline-2-carboxylic acid 1-aza-bicyclo[2.2.2]oct-3-yl ester) and mesylate 5 as starting materials.

### Example 40

### 1-[5-(2-tert-Butylamino-1-hydroxy-ethyl)-2-phosphonooxymethoxy-benzyl]-3-(1-cyclohexyl-3,4-dihydro-1H-isoquinoline-2-carbonyloxy)-1-azonia-bicyclo[2.2.2]octane

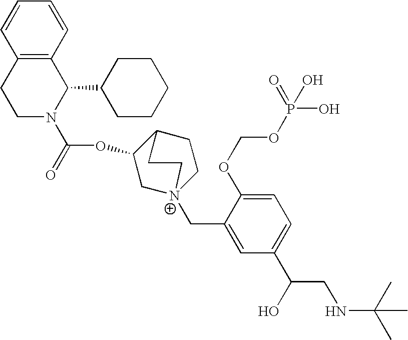

The title compound can be prepared through a two-step procedure described in Example 29 applied to Solifenacin (1-Cyclohexyl-3,4-dihydro-1H-isoquinoline-2-carboxylic acid 1-aza-bicyclo[2.2.2]oct-3-yl ester) and mesylate 13 as starting materials.

### Example 41

### 3-(2-Hydroxymethyl-4-methanesulfinyl-2-phenyl-butyryloxy)-1-(5-{1-hydroxy-2-[6-(4-phenyl-butoxy)-hexylamino]-ethyl}-2-phosphonooxy-benzyl)-1-azonia-bicyclo[2.2.2]octane

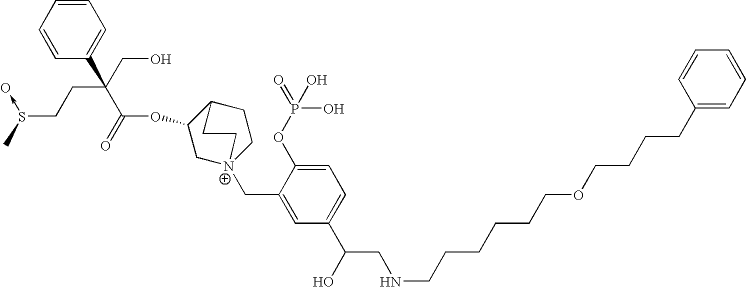

The title compound can be prepared through a two-step procedure described in Example 29 applied to Revatropate (2-Hydroxymethyl-4-methanesulfinyl-2-phenyl-butyric acid 1-aza-bicyclo[2.2.2]oct-3-yl ester) and mesylate 3 as starting materials.

### Example 42

### 1-[5-(2-tert-Butylamino-1-hydroxy-ethyl)-2-phosphonooxy-benzyl]-3-(2-hydroxymethyl-4-methanesulfinyl-2-phenyl-butyryloxy)-1-azonia-bicyclo[2.2.2]octane

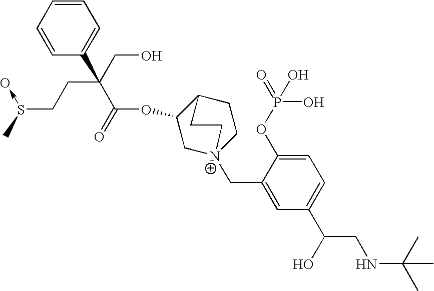

The title compound can be prepared through a two-step procedure described in Example 29 applied to Revatropate (2-Hydroxymethyl-4-methanesulfinyl-2-phenyl-butyric acid 1-aza-bicyclo[2.2.2]oct-3-yl ester) and mesylate 10 as starting materials.

### Example 43

### 3-(2-Hydroxymethyl-4-methanesulfinyl-2-phenyl-butyryloxy)-1-(5-{1-hydroxy-2-[6-(4-phenyl-butoxy)-hexylamino]-ethyl}-2-phosphonooxymethoxy-benzyl)-1-azonia-bicyclo[2.2.2]octane

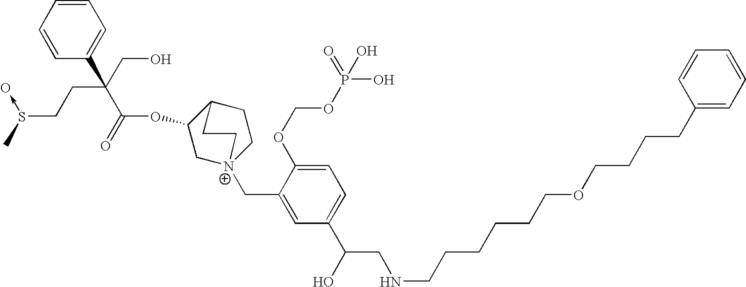

The title compound can be prepared through a two-step procedure described in Example 29 applied to Revatropate (2-Hydroxymethyl-4-methanesulfinyl-2-phenyl-butyric acid 1-aza-bicyclo[2.2.2]oct-3-yl ester) and mesylate 5 as starting materials.

### Example 44

### 1-[5-(2-tert-Butylamino-1-hydroxy-ethyl)-2-phosphonooxymethoxy-benzyl]-3-(2-hydroxymethyl-4-methanesulfinyl-2-phenyl-butyryloxy)-1-azonia-bicyclo[2.2.2]octane

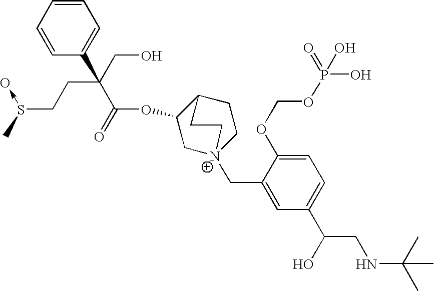

The title compound can be prepared through a two-step procedure described in Example 29 applied to Revatropate (2-Hydroxymethyl-4-methanesulfinyl-2-phenyl-butyric acid 1-aza-bicyclo[2.2.2]oct-3-yl ester) and mesylate 13 as starting materials.

### Example 45

### 3-(Carbamoyl-diphenyl-methyl)-1-[2-(2,3-dihydro-benzofuran-5-yl)-ethyl]-1-(5-{1-hydroxy-2-[6-(4-phenyl-butoxy)-hexylamino]-ethyl}-2-phosphonooxy-benzyl)-pyrrolidinium

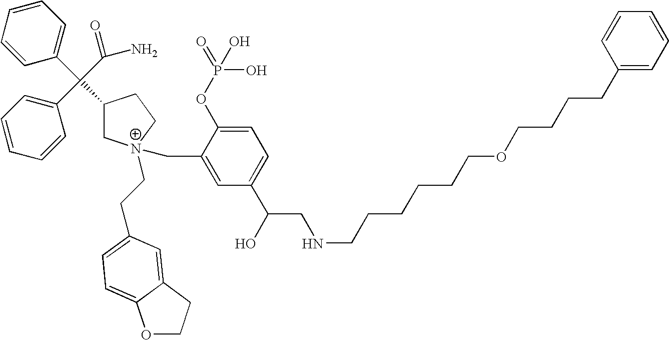

The title compound can be prepared through a two-step procedure described in Example 29 applied to Darifenacin (2-{1-[2-(2,3-Dihydro-benzofuran-5-yl)-ethyl]-pyrrolidin-3-yl}-2,2-diphenyl-acetamide) and mesylate 3 as starting materials.

### Example 46

### 1-[5-(2-tert-Butylamino-1-hydroxy-ethyl)-2-phosphonooxy-benzyl]-3-(carbamoyl-diphenyl-methyl)-1-[2-(2,3-dihydro-benzofuran-5-yl)-ethyl]-pyrrolidinium

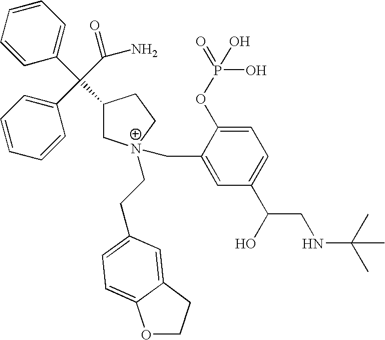

The title compound can be prepared through a two-step procedure described in Example 29 applied to Darifenacin (2-{1-[2-(2,3-Dihydro-benzofuran-5-yl)-ethyl]-pyrrolidin-3-yl}-2,2-diphenyl-acetamide) and mesylate 10 as starting materials.

### Example 47

### 3-(Carbamoyl-diphenyl-methyl)-1-[2-(2,3-dihydro-benzofuran-5-yl)-ethyl]-1-(5-{1-hydroxy-2-[6-(4-phenyl-butoxy)-hexylamino]-ethyl}-2-phosphonooxymethoxy-benzyl)-pyrrolidinium

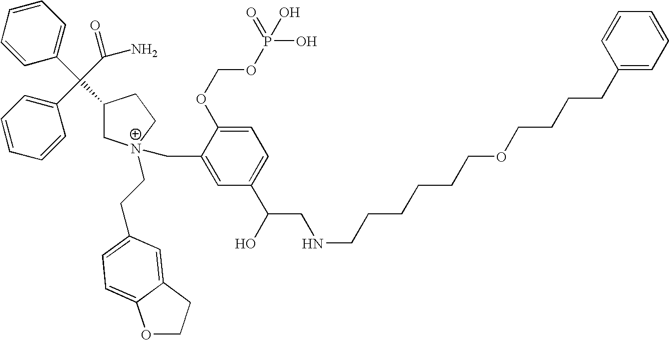

The title compound can be prepared through a two-step procedure described in Example 29 applied to Darifenacin (2-{1-[2-(2,3-Dihydro-benzofuran-5-yl)-ethyl]-pyrrolidin-3-yl}-2,2-diphenyl-acetamide) and mesylate 5 as starting materials.

### Example 48

### 1-[5-(2-tert-Butylamino-1-hydroxy-ethyl)-2-phosphonooxymethoxy-benzyl]-3-(carbamoyl-diphenyl-methyl)-1-[2-(2,3-dihydro-benzofuran-5-yl)-ethyl]-pyrrolidinium

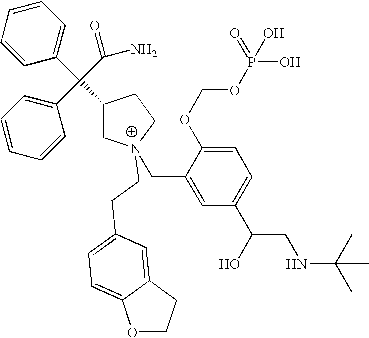

The title compound can be prepared through a two-step procedure described in Example 29 applied to Darifenacin (2-{1-[2-(2,3-Dihydro-benzofuran-5-yl)-ethyl]-pyrrolidin-3-yl}-2,2-diphenyl-acetamide) and mesylate 13 as starting materials.

### Example 49

### 1-(3-Carbamoyl-3,3-diphenyl-propyl)-1-(5-{1-hydroxy-2-[6-(4-phenyl-butoxy)-hexylamino]-ethyl}-2-phosphonooxy-benzyl)-azepanium

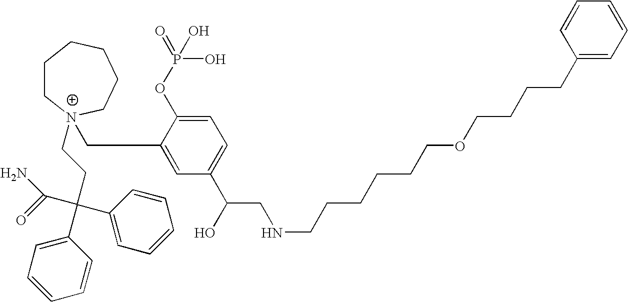

The title compound can be prepared through a two-step procedure described in Example 29 applied to Buzepide (4-azepan-1-yl-2,2-diphenyl-butyramide) and mesylate 3 as starting materials.

### Example 50

### 1-[5-(2-tert-Butylamino-1-hydroxy-ethyl)-2-phosphonooxy-benzyl]-1-(3-carbamoyl-3,3-diphenyl-propyl)-azepanium

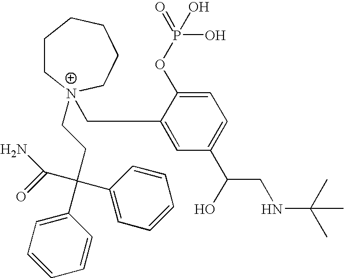

The title compound can be prepared through a two-step procedure described in Example 29 applied to Buzepide (4-azepan-1-yl-2,2-diphenyl-butyramide) and mesylate 10 as starting materials.

### Example 51

### 1-(3-Carbamoyl-3,3-diphenyl-propyl)-1-(5-{1-hydroxy-2-[6-(4-phenyl-butoxy)-hexylamino]-ethyl}-2-phosphonooxymethoxy-benzyl)-azepanium

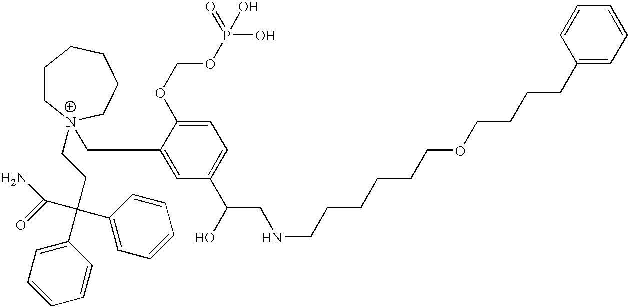

The title compound can be prepared through a two-step procedure described in Example 29 applied to Buzepide (4-azepan-1-yl-2,2-diphenyl-butyramide) and mesylate 5 as starting materials.

### Example 52

### 1-[5-(2-tert-Butylamino-1-hydroxy-ethyl)-2-phosphonooxymethoxy-benzyl]-1-(3-carbamoyl-3,3-diphenyl-propyl)-azepanium

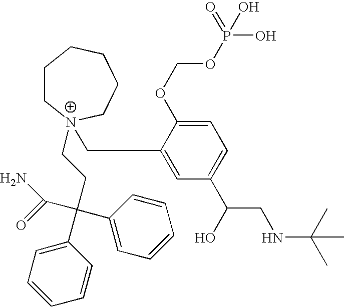

The title compound can be prepared through a two-step procedure described in Example 29 applied to Buzepide (4-azepan-1-yl-2,2-diphenyl-butyramide) and mesylate 13 as starting materials.

### Example 53

### Phosphono-Salmeterol-Oxitropium-N,N-diethylglycinate

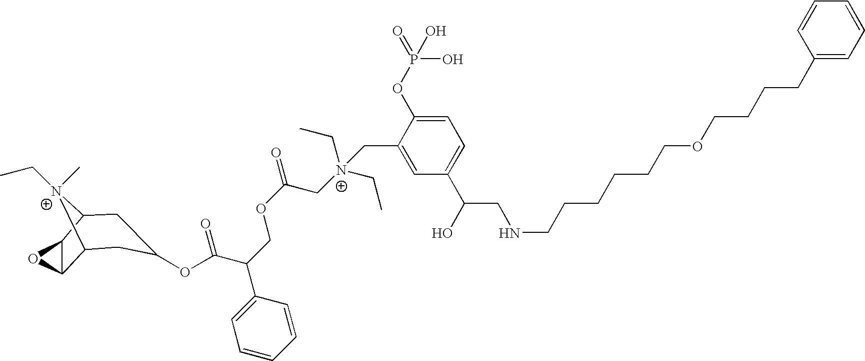

Oxitropium (9-Ethyl-7-(3-hydroxy-2-phenyl-propionyloxy)-9-methyl-3-oxa-9-azonia-tricyclo[3.3.1.02,4]nonane) can be esterifed with N,N-diethylglycine according to the procedure described in Example 33, yielding 7-[3-(2-Diethylamino-acetoxy)-2-phenyl-propionyloxy]-9-ethyl-9-methyl-3-oxa-9-azonia-tricyclo[3.3.1.02,4]nonane.

The title compound can be prepared through a two-step procedure described in Example 29 applied to 7-[3-(2-Diethylamino-acetoxy)-2-phenyl-propionyloxy]-9-ethyl-9-methyl-3-oxa-9-azonia-tricyclo[3.3.1.02,4]nonane and mesylate 3 as starting materials.

### Example 54

### Phosphono-Albuterol-Oxitropium-N,N-diethylglyciate

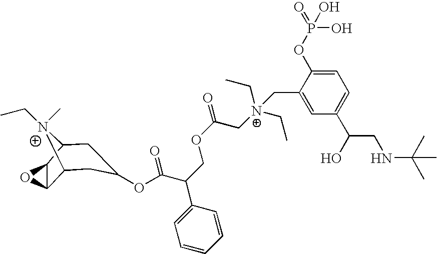

The title compound can be prepared through a two-step procedure described in Example 29 applied to 7-[3-(2-Diethylamino-acetoxy)-2-phenyl-propionyloxy]-9-ethyl-9-methyl-3-oxa-9-azonia-tricyclo[3.3.1.02,4]nonane and mesylate 10 as starting materials.

### Example 55

### Phosphonooxymethylene-Salmeterol-Oxitropium-N,N-diethylglycinate

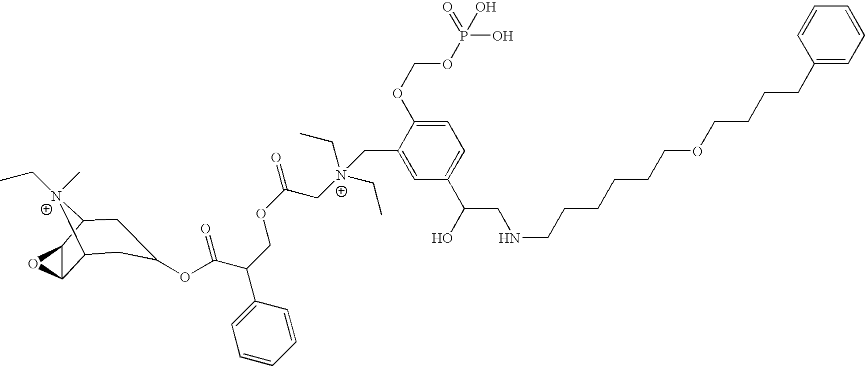

The title compound can be prepared through a two-step procedure described in Example 29 applied to 7-[3-(2-Diethylamino-acetoxy)-2-phenyl-propionyloxy]-9-ethyl-9-methyl-3-oxa-9-azonia-tricyclo[3.3.1.02,4]nonane and mesylate 5 as starting materials.

### Example 56

### Phosphonooxymethylene-Albuterol-Oxitronium-N,N-diethylglycinate

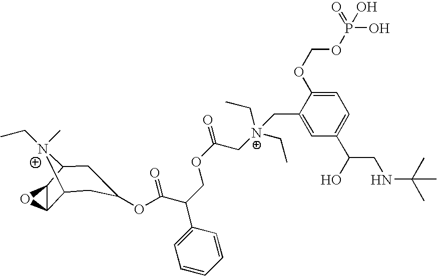

The title compound can be prepared through a two-step procedure described in Example 29 applied to 7-[3-(2-Diethylamino-acetoxy)-2-phenyl-propionyloxy]-9-ethyl-9-methyl-3-oxa-9-azonia-tricyclo[3.3.1.02,4]nonane and mesylate 13 as starting materials.

### Example 57

### Phosphono-Salmeterol-Tiotropium-N,N-diethylglycinate

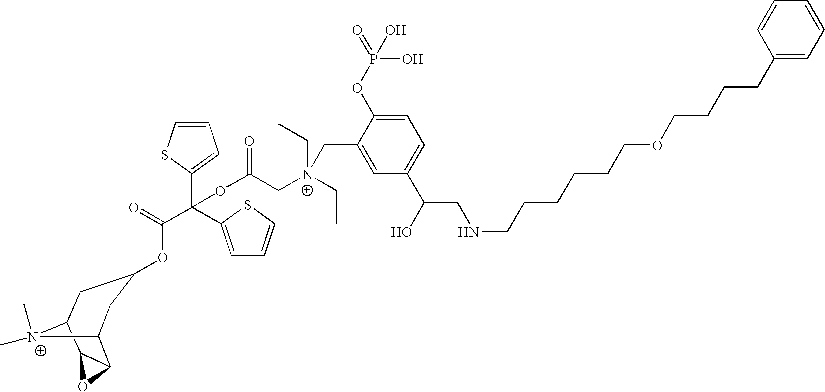

Tiotropium [7-(2-Hydroxy-2,2-di-thiophen-2-yl-acetoxy)-9,9-dimethyl-3-oxa-9-azonia-tricyclo[3.3.1.02,4]nonane] can be esterified with N,N-diethylglycine according to the procedure described in Example 33, yielding 7-[2-(2-Diethylamino-acetoxy)-2,2-di-thiophen-2-yl-acetoxy]-9,9-dimethyl-3-oxa-9-azonia-tricyclo[3.3.1.02,4]nonane.

The title compound can be prepared through a two-step procedure described in Example 29 applied to 7-[2-(2-Diethylamino-acetoxy)-2,2-di-thiophen-2-yl-acetoxy]-9,9-dimethyl-3-oxa-9-azonia-tricyclo[3.3.1.02,4]nonane and mesylate 3 as starting materials.

### Example 58

### Phosphono-Albuterol-Tiotropium-N,N-diethylglycinate

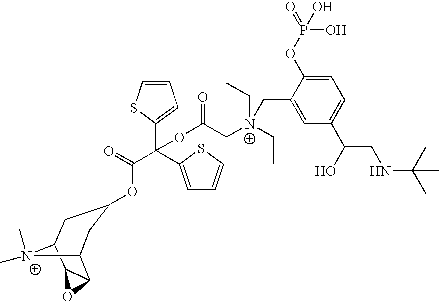

The title compound can be prepared through a two-step procedure described in Example 29 applied to 7-[2-(2-Diethylamino-acetoxy)-2,2-di-thiophen-2-yl-acetoxy]-9,9-dimethyl-3-oxa-9-azonia-tricyclo[3.3.1.02,4]nonane and mesylate 10 as starting materials.

### Example 59

### Phosphonooxymethylene-Salmeterol-Tiotropium-N,N-diethylglycinate

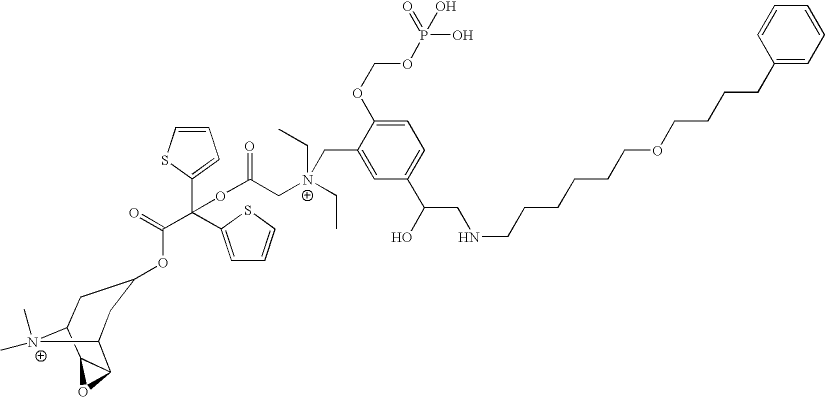

The title compound can be prepared through a two-step procedure described in Example 29 applied to 7-[2-(2-Diethylamino-acetoxy)-2,2-di-thiophen-2-yl-acetoxy]-9,9-dimethyl-3-oxa-9-azonia-tricyclo[3.3.1.02,4]nonane and mesylate 5 as starting materials.

### Example 60

### Phosphonooxymethylene-Albuterol-Tiotropium-N,N-diethylglycinate

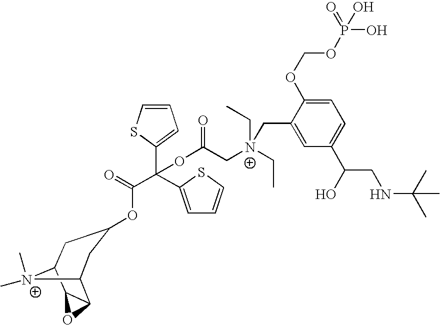

The title compound can be prepared through a two-step procedure described in Example 29 applied to 7-[2-(2-Diethylamino-acetoxy)-2,2-di-thiophen-2-yl-acetoxy]-9,9-dimethyl-3-oxa-9-azonia-tricyclo[3.3.1.02,4]nonane and mesylate 13 as starting materials.

### Example 61

### [2-(3-Diisopropylamino-1-phenyl-propyl)-4-methyl-phenoxycarbonylmethyl]-(5-{1-hydroxy-2-[6-(4-phenyl-butoxy)-hexylamino]-ethyl}-2-phosphonooxy-benzyl)-dimethyl-ammonium

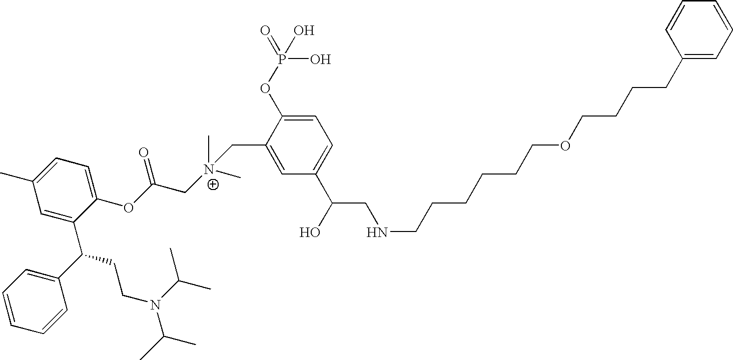

Tolterodine [2-(3-Diisopropylamino-1-phenyl-propyl)-4-methyl-phenol] can be esterified with N,N-dimethylglycine according to the procedure described in Example 33, yielding dimethylamino-acetic acid 2-(3-diisopropylamino-1-phenyl-propyl)-4-methyl-phenyl ester.

The title compound can be prepared through a two-step procedure described in Example 29 applied to dimethylamino-acetic acid 2-(3-diisopropylamino-1-phenyl-propyl)-4-methyl-phenyl ester and mesylate 3 as starting materials.

### Example 62

### [5-(2-tert-Butylamino-1-hydroxy-ethyl)-2-phosphonooxy-benzyl]-[2-(3-diisopropylamino-1-phenyl-propyl)-4-methyl-phenoxycarbonylmethyl]-dimethyl-ammonium

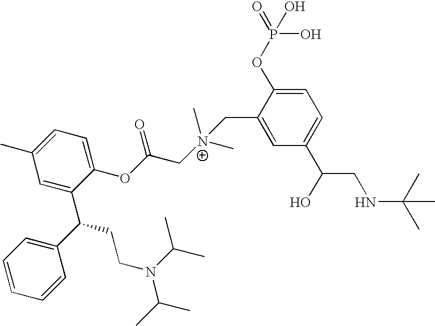

The title compound can be prepared through a two-step procedure described in Example 29 applied to dimethylamino-acetic acid 2-(3-diisopropylamino-1-phenyl-propyl)-4-methyl-phenyl ester and mesylate 10 as starting materials.

### Example 63

### [2-(3-Diisopropylamino-1-phenyl-propyl)-4-methyl-phenoxycarbonylmethyl]-(5-{1-hydroxy-2-[6-(4-phenyl-butoxy)-hexylamino]-ethyl}-2-phosphonooxymethoxy-benzyl)-dimethyl-ammonium

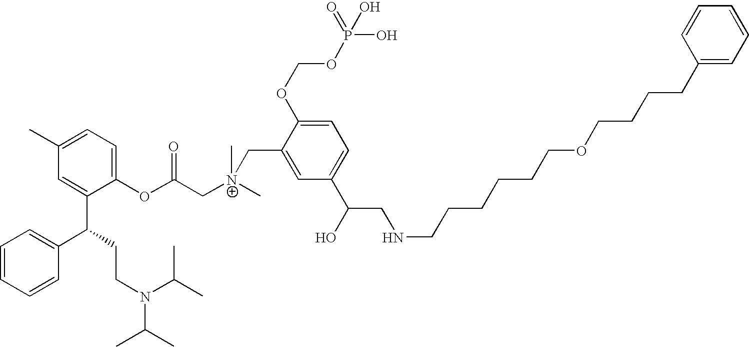

The title compound can be prepared through a two-step procedure described in Example 29 applied to dimethylamino-acetic acid 2-(3-diisopropylamino-1-phenyl-propyl)-4-methyl-phenyl ester and mesylate 5 as starting materials.

### Example 64

### [5-(2-tert-Butylamino-1-hydroxy-ethyl)-2-phosphonooxymethoxy-benzyl]-[2-(3-diisopropylamino-1-phenyl-propyl)-4-methyl-phenoxycarbonylmethyl]-dimethyl-ammonium

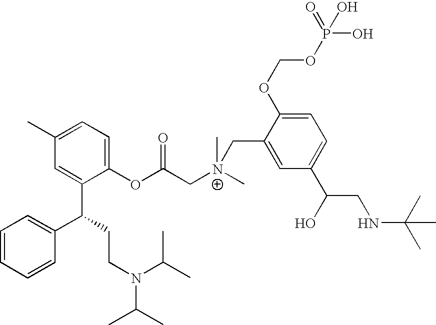

The title compound can be prepared through a two-step procedure described in Example 29 applied to dimethylamino-acetic acid 2-(3-diisopropylamino-1-phenyl-propyl)-4-methyl-phenyl ester and mesylate 13 as starting materials.

### Example 65

### Phosphono-Salmeterol-3-[4,4-Bis-(4-fluoro-phenyl)-2-oxo-imidazolidin-1-yl]-1-methyl-1-(2-oxo-2-pyridin-2-yl-ethyl)-pyrrolidinium

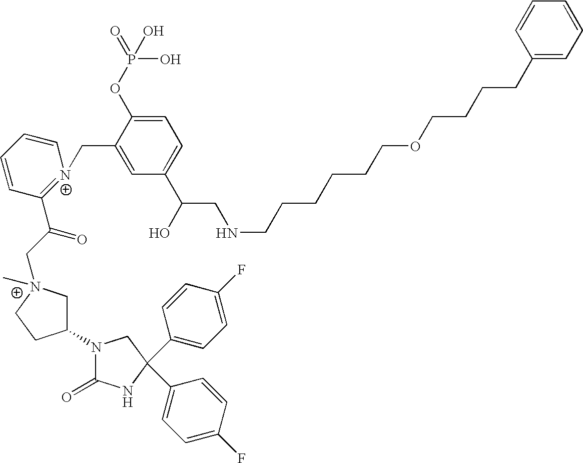

The title compound can be prepared through a two-step procedure described in Example 29 applied to 3-[4,4-bis-(4-fluoro-phenyl)-2-oxo-imidazolidin-1-yl]-1-methyl-1-(2-oxo-2-pyridin-2-yl-ethyl)-pyrrolidinium (prepared according to Peretto et al., 2007, Part 2) and mesylate 3 as starting materials.

### Example 66

### Phosphono-Albuterol-3-[4,4-Bis-(4-fluoro-phenyl)-2-oxo-imidazolidin-1-yl]-1-methyl-1-(2-oxo-2-pyridin-2-yl-ethyl)-pyrrolidinium

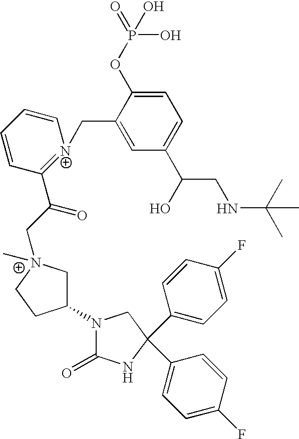

The title compound can be prepared through a two-step procedure described in Example 29 applied to 3-[4,4-bis-(4-fluoro-phenyl)-2-oxo-imidazolidin-1-yl]-1-methyl-1-(2-oxo-2-pyridin-2-yl-ethyl)-pyrrolidinium and mesylate 10 as starting materials.

### Example 67

### Phosphonooxymethylene-Salmeterol-3-[4,4-Bis-(4-fluoro-phenyl)-2-oxo-imidazolidin-1-yl]-1-methyl-1-(2-oxo-2-pyridin-2-ethyl)-pyrrolidinium

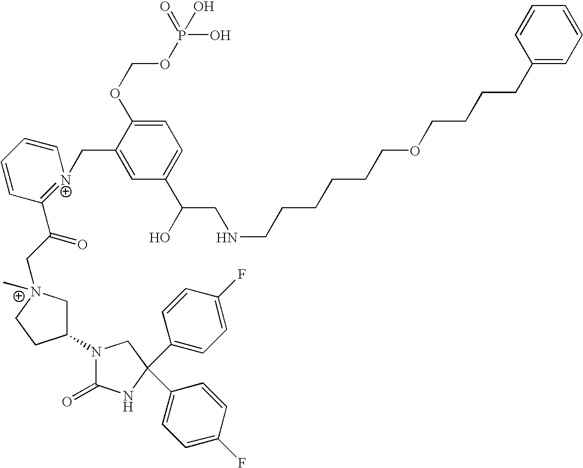

The title compound can be prepared through a two-step procedure described in Example 29 applied to 3-[4,4-bis-(4-fluoro-phenyl)-2-oxo-imidazolidin-1-yl]-1-methyl-1-(2-oxo-2-pyridin-2-yl-ethyl)-pyrrolidinium and mesylate 5 as starting materials.

### Example 68

### Phosphonooxymethylene-Albuterol-3-[4,4-Bis-(4-fluoro-phenyl)-2-oxo-imidazolidin-1-yl]-1-methyl-1-(2-oxo-2-pyridin-2-yl-ethyl)-pyrrolidinium

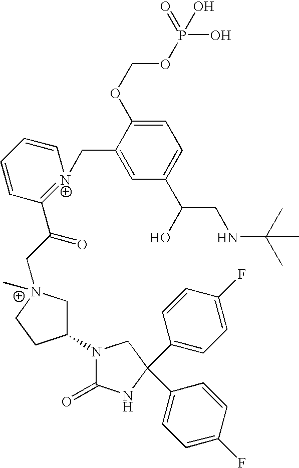

The title compound can be prepared through a two-step procedure described in Example 29 applied to 3-[4,4-bis-(4-fluoro-phenyl)-2-oxo-imidazolidin-1-yl]-1-methyl-1-(2-oxo-2-pyridin-2-yl-ethyl)-pyrrolidinium and mesylate 13 as starting materials.

### Example 69

### 4-[4,4-Bis-(4-fluoro-phenyl)-2-oxo-imidazolidin-1-yl]-1-(3-fluoro-benzyl)-1-(5-{1-hydroxy-2-[6-(4-phenyl-butoxy)-hexylamino]-ethyl}-2-phosphonooxy-benzyl)-piperidinium

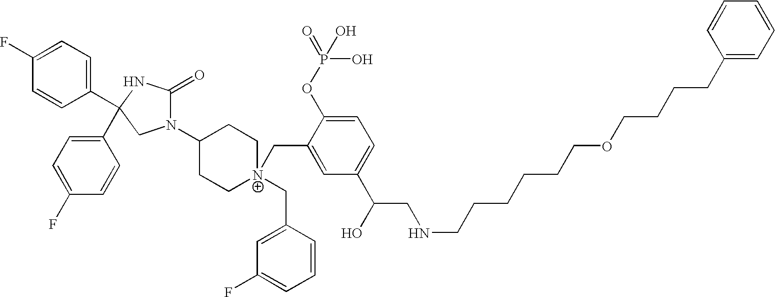

The title compound can be prepared through a two-step procedure described in Example 29 applied to 1-[1-(3-fluoro-benzyl)-piperidin-4-yl]-4,4-bis-(4-fluoro-phenyl)-imidazolidin-2-one (prepared according to Peretto et al., 2007, Part 1) and mesylate 3 as starting materials.

### Example 70

### 4-[4,4-Bis-(4-fluoro-phenyl)-2-oxo-imidazolidin-1-yl]-1-[5-(2-tert-butylamino-1-hydroxy-ethyl)-2-phosphonooxy-benzyl]-1-(3-fluoro-benzyl)-piperidinium

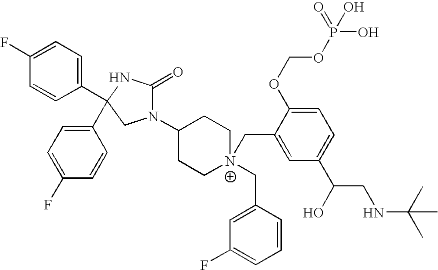

The title compound can be prepared through a two-step procedure described in Example 29 applied to 1-[1-(3-fluoro-benzyl)-piperidin-4-yl]-4,4-bis-(4-fluoro-phenyl)-imidazolidin-2-one and mesylate 10 as starting materials.

### Example 71

### 4-[4,4-Bis-(4-fluoro-phenyl)-2-oxo-imidazolidin-1-yl]-1-(3-fluoro-benzyl)-1-(5-{1-hydroxy-2-[6-(4-phenyl-butoxy)-hexylamino]-ethyl}-2-phosphonooxymethyloxy-benzyl)-piperidinium

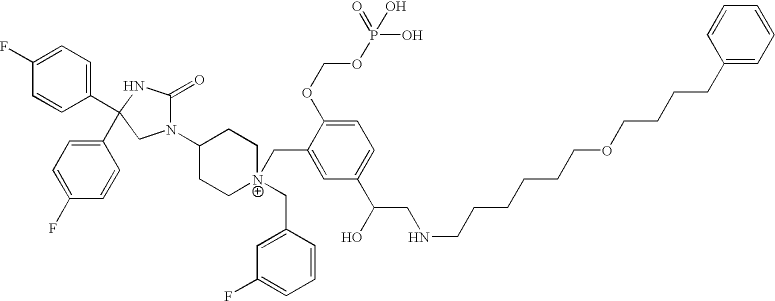

The title compound can be prepared through a two-step procedure described in Example 29 applied to 1-[1-(3-fluoro-benzyl)-piperidin-4-yl]-4,4-bis-(4-fluoro-phenyl)-imidazolidin-2-one and mesylate 10 as starting materials.

### Example 72

### 4-[4,4-Bis-(4-fluoro-phenyl)-2-oxo-imidazolidin-1-yl]-1-[5-(2-tert-butylamino-1-hydroxyethyl)-2-phosphonooxymethoxy-benzyl]-1-(3-fluoro-benzyl)-piperidinium

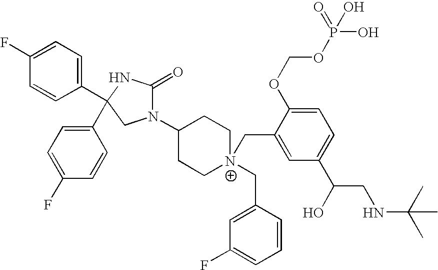

The title compound can be prepared through a two-step procedure described in Example 29 applied to 1-[1-(3-fluoro-benzyl)-piperidin-4-yl]-4,4-bis-(4-fluoro-phenyl)-imidazolidin-2-one and mesylate 13 as starting materials.

### Example 73

### 3-(3-Cyclooctyl-3-hydroxy-3-phenyl-prop-1-ynyl)-1-(5-{1-hydroxy-2-[6-(4-phenyl-butoxy)-hexylamino]-ethyl}-2-phosphonooxy-benzyl)-3-methoxy-1-azonia-bicyclo[2.2.2]octane

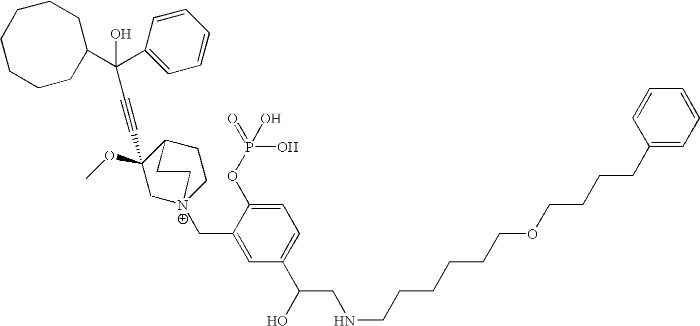

The title compound can be prepared through a two-step procedure described in Example 29 applied to 1-cyclooctyl-3-(3-methoxy-1-aza-bicyclo[2.2.2]oct-3-yl)-1-phenyl-prop-2-yn-1-ol (prepared as described by Provins et al., 2006) and mesylate 3 as starting materials.

### Example 74

### 1-[5-(2-tert-Butylamino-1-hydroxy-ethyl)-2-phosphonooxy-benzyl]-3-(3-cyclooctyl-3-hydroxy-3-phenyl-prop-1-ynyl)-3-methoxy-1-azonia-bicyclo[2.2.2]octane

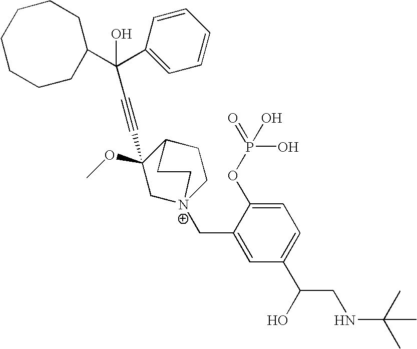

The title compound can be prepared through a two-step procedure described in Example 29 applied to 1-cyclooctyl-3-(3-methoxy-1-aza-bicyclo[2.2.2]oct-3-yl)-1-phenyl-prop-2-yn-1-ol and mesylate 10 as starting materials.

### Example 75

### 3-(3-Cyclooctyl-3-hydroxy-3-phenyl-prop-1-ynyl)-1-(5-{1-hydroxy-2-[6-(4-phenyl-butoxy)-hexylamino]-ethyl}-2-phosphonooxymethoxy-benzyl)-3-methoxy-1-azonia-bicyclo[2.2.2]octane

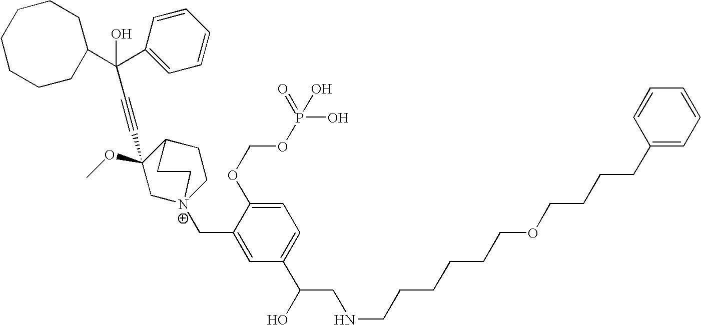

The title compound can be prepared through a two-step procedure described in Example 29 applied to 1-cyclooctyl-3-(3-methoxy-1-aza-bicyclo[2.2.2]oct-3-yl)-1-phenyl-prop-2-yn-1-ol and mesylate 5 as starting materials.

### Example 76

### 1-[5-(2-tert-Butylamino-1-hydroxy-ethyl)-2-phosphonooxymethoxy-benzyl]-3-(3-cyclooctyl-3-hydroxy-3-phenyl-prop-1-ynyl)-3-methoxy-1-azonia-bicyclo[2.2.2]octane

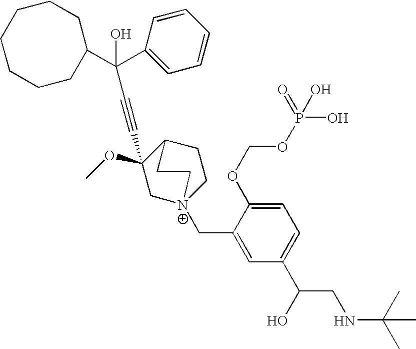

The title compound can be prepared through a two-step procedure described in Example 29 applied to 1-cyclooctyl-3-(3-methoxy-1-aza-bicyclo[2.2.2]oct-3-yl)-1-phenyl-prop-2-yn-1-ol and mesylate 13 as starting materials.

### Example 77

### Phosphono-Salmeterol-3-[2-(2-Diethylamino-acetoxy)-2,2-di-thiophen-2-yl-acetoxy]-1-(3-phenoxy-propyl)-1-azonia-bicyclo[2.2.2]octane

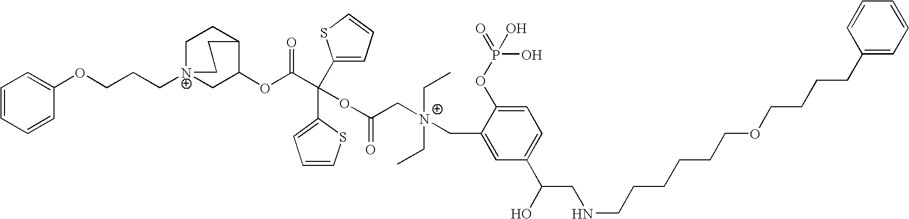

3-(2-Hydroxy-2,2-di-thiophen-2-yl-acetoxy)-1-(3-phenoxy-propyl)-1-azonia-bicyclo [2.2.2]octane (Aclidinium, described in US Patent by Meade et al., 2005) was esterified with N,N-diethylglycine according to the procedure described in Example 33, yielding 3-[2-(2-Diethylamino-acetoxy)-2,2-di-thiophen-2-yl-acetoxy]-1-(3-phenoxy-propyl)-1-azonia-bicyclo[2.2.2]octane.

The title compound can be prepared through a two-step procedure described in Example 29 applied to 3-[2-(2-Di ethylamino-acetoxy)-2,2-d i-thiophen-2-yl-acetoxy]-1-(3-phenoxy-propyl)-1-azonia-bicyclo[2.2.2]octane and mesylate 3 as starting materials.

### Example 78

### Phosphono-Albuterol-3-[2-(2-Diethylamino-acetoxy)-2,2-di-thiophen-2-yl-acetoxy]-1-(3-phenoxy-propyl)-1-azonia-bicyclo[2.2.2]octane

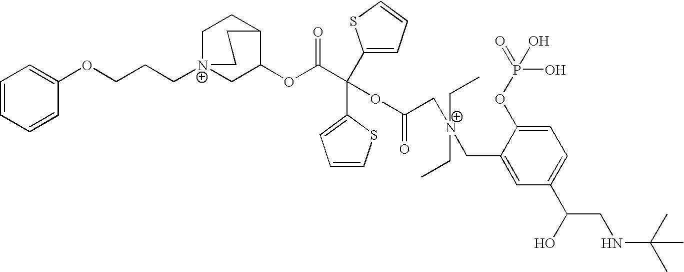

The title compound can be prepared through a two-step procedure described in Example 29 applied to 3-[2-(2-Diethylamino-acetoxy)-2,2-di-thiophen-2-yl-acetoxy]-1-(3-phenoxy-propyl)-1-azonia-bicyclo[2.2.2]octane and mesylate 10 as starting materials.

### Example 79

### Phosphonooxymethylene-Salmeterol-3-[2-(2-Diethylamino-acetoxy)-2,2-di-thiophen-2-yl-acetoxy]-1-(3-phenoxy-propyl)-1-azonia-bicyclo[2.2.2]octane

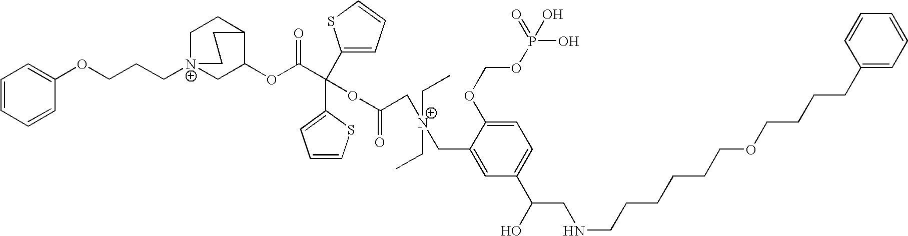

The title compound can be prepared through a two-step procedure described in Example 29 applied to 3-[2-(2-Diethylamino-acetoxy)-2,2-di-thiophen-2-yl-acetoxy]-1-(3-phenoxy-propyl)-1-azonia-bicyclo[2.2.2]octane and mesylate 5 as starting materials.

### Example 80

### Phosphonooxymethylene-Albuterol-3-[2-(2-Diethylamino-acetoxy)-2,2-di-thiophen-2-yl-acetoxy]-1-(3-phenoxy-propyl)-1-azonia-bicyclo[2.2.2]octane

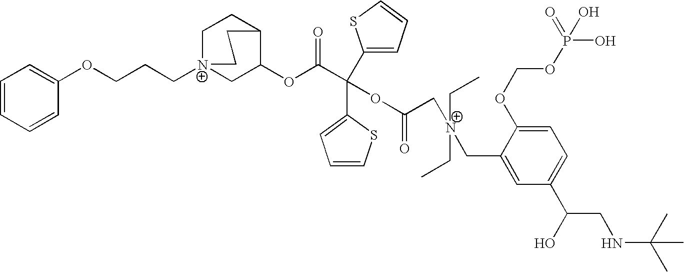

The title compound can be prepared through a two-step procedure described in Example 29 applied to 3-[2-(2-Diethylamino-acetoxy)-2,2-di-thiophen-2-yl-acetoxy]-1-(3-phenoxy-propyl)-1-azonia-bicyclo[2.2.2]octane and mesylate 13 as starting materials.

### Example 81

### Diethyl-(5-{1-hydroxy-2-[6-(4-phenyl-butoxy)-hexylamino]-ethyl}-2-bhosphonooxybenzyl)-{[1-methyl-1-(2-phenoxy-ethyl)-piperidin-4-yloxycarbonyl]-di-thiophen-2-yl-methoxycarbonylmethyl}-ammonium

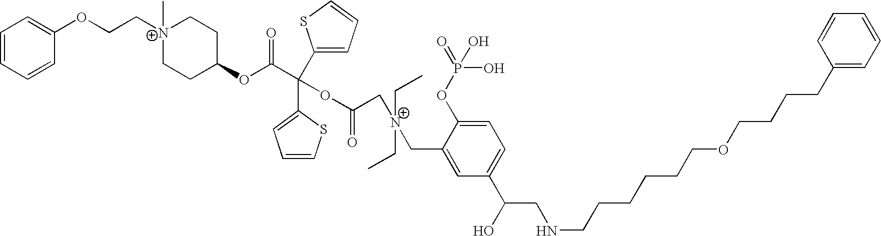

4-(2-Hydroxy-2,2-di-thiophen-2-yl-acetoxy)-1-methyl-1-(2-phenoxy-ethyl)-piperidinium (prepared as described by Baettig et al., 2007) was esterified with N,N-diethylglycine according to the procedure described in Example 33, yielding (2-diethylamino-acetoxy)-di-thiophen-2-yl-acetic acid 1-methyl-1-(2-phenoxy-ethyl)-piperidin-4-yl ester.

The title compound can be prepared through a two-step procedure described in Example 29 applied to (2-diethylamino-acetoxy)-di-thiophen-2-yl-acetic acid 1-methyl-1-(2-phenoxy-ethyl)-piperidin-4-yl ester and mesylate 3 as starting materials.

### Example 82

### [5-(2-tent-Butylamino-1-hydroxy-ethyl)-2-phosphonooxy-benzyl]-diethyl-{[1-methyl-1-(2-phenoxy-ethyl)-piperidin-4-yloxycarbonyl]-di-thiophen-2-yl-methoxycarbonylmethyl}-ammonium

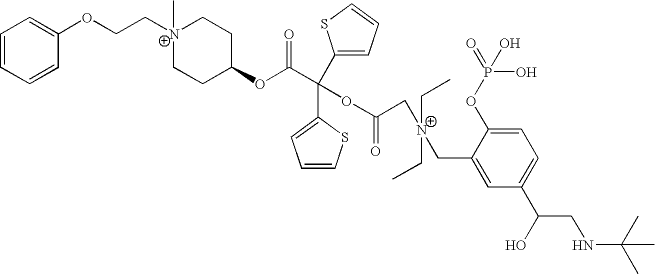

The title compound can be prepared through a two-step procedure described in Example 29 applied to (2-diethylamino-acetoxy)-di-thiophen-2-yl-acetic acid 1-methyl-1-(2-phenoxy-ethyl)-piperidin-4-yl ester and mesylate 10 as starting materials.

### Example 83

### Diethyl-(5-{1-hydroxy-2-[6-(4-phenyl-butoxy)-hexylamino]-ethyl}-2-phosphonooxymethyleneoxy-benzyl)-{[1-methyl-1-(2-phenoxy-ethyl)-piperidin-4-yloxycarbonyl]-di-thiophen-2-yl-methoxycarbonylmethyl}-ammonium

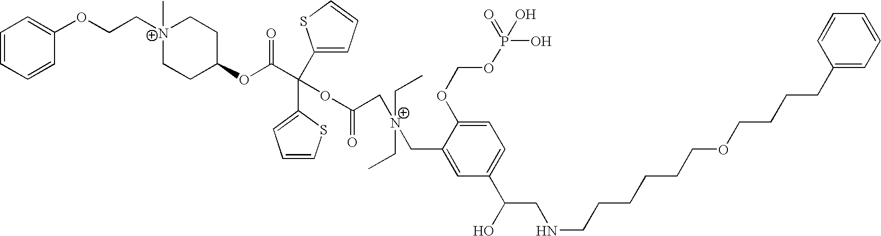

The title compound can be prepared through a two-step procedure described in Example 29 applied to (2-diethylamino-acetoxy)-di-thiophen-2-yl-acetic acid 1-methyl-1-(2-phenoxy-ethyl)-piperidin-4-yl ester and mesylate 5 as starting materials.

### Example 84

### [5-(2-tert-Butylamino-1-hydroxy-ethyl)-2-phosphonooxymethoxy-benzyl]-diethyl-{1-methyl-1-(2-phenoxy-ethyl)-piperidin-4-yloxycarbonyl]-di-thiophen-2-yl-methoxycarbonylmethyl}-ammonium

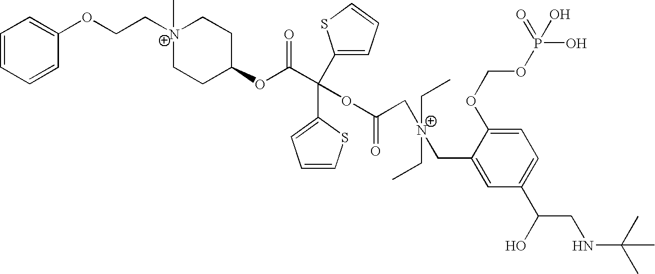

The title compound can be prepared through a two-step procedure described in Example 29 applied to (2-diethylamino-acetoxy)-di-thiophen-2-yl-acetic acid 1-methyl-1-(2-phenoxy-ethyl)-piperidin-4-yl ester and mesylate 13 as starting materials.

### Example 85

### Conversion of the Mutual MRA-β-Agonist Prodrug (Described in Example 29) to Salmeterol and MRA after Exposure to Alkaline Phosphatase In Vitro

Preparation of Stock Solutions:

50 mM pH 7.4 Tris Buffer Stock Solution

Dissolved 1.500 g (12.5 mmol) tris(hydroxymethyl)aminomethane in ˜200 ml water, added ˜1600 μl of 6M HCl, diluted to 250 ml with water. Final pH=7.45 (measured using a Thermo Orion ROSS pH electrode). Stored at 2°-8° C.,

50 mM MgCl₂ Stock Solution

Dissolved 2.033 g (10 mmol) MgCl₂ 6H₂O in 200 ml water to form 50 mM of MgCl₂ solution. Stored at 2°-8° C.

50 mM ZnCl₂ Stock Solution

Dissolved 1.364 g (10 mmol) of ZnCl₂ in 200 ml water. About 0.1 mL of 6 M HCl was added into solution to dissolve insoluble Zn carbonate or hydroxide. Store at 2°-8° C.

Reaction Buffer (pH 7.4, 5 mM tris/1 mM Mg²⁺/1 mM Zn²⁺)

Diluted 5 ml of 50 mM tris stock, 1 ml of 50 mM MgCl₂ stock, and 1 ml of ZnCl₂ and then stocked to 10 ml with water.

Alkaline Phosphatase Stock Solution

Dispersed ˜1 mg (pre-weight) of Sigma P-3895 alkaline phosphatase (Lot number 023K37902) in reaction buffer to make the final concentration of 0.224 mg/mL.

Prodrug Stock Solution

Dissolved ˜2 mg of the mutual prodrug of invention in 10 ml 1:1 acetonitrile/water.

Reaction Product Stock Solution

Dissolved ˜2 mg of each MRA and β-agonist in 20 ml 1:1 acetonitrile/water

Reaction Procedure

The stock solutions were mixed in microcentrifuge tubes, as depicted in the following Table:

| | | Alkaline | Drug | Reaction | 1:1 aq. |
|---|---|---|---|---|---|
| Solution | Prodrug | phosphatase | standards | buffer | AcN |
| Blank | — | — | — | 500 μl | 500 μl |
| Drug standards | — | — | 500 μl | 500 μl | — |
| Prodrug | 500 μl | — | — | 500 μl | — |
| Reaction | 500 μl | 500 μl | 0 | 0 | — |

The heat block was set at the 37 degrees. Then 0.5 mL of alkaline phosphatase solution was added into 4 preheated Eppendorf tubes. The aliquot 0.5 of prodrug and drug standards were added into preheated Eppendorf tubes. Immediately after vortexing the aliquots of 25 μL of the all reaction solutions were made into the respective 96-well plate positions. The internal standard (75 μl of 500 ng/mL Glyburide) was added into all samples after each aliquots. That procedure was repeated at every 15 minute intervals for ˜4-5 hrs.

The 96-well plates were then analyzed using the LCMS technique.

HPLC-MS parameters (typical)
LC Gradient

| Run time: | 3.0 min |
|---|---|
| Column Flow: | 0.500 ml/min |
| Gradient | |
| Time (min) | % B |
| 0-0.30 | 15 |
| 1.50 | 95 |
| 2.30 | 95 |
| 2.40 | 15 |
| 3.00 | 15 |
| Mobile Phase A: | 1% formic acid in water |
| Mobile Phase B: | 1% formic acid in acetonitrile |

Autosampler

| | |
|---|---|
| Injection Volume: | 5.0 μl |
| Autosample Tray Temperature: | 5 ± 3° C. |

Column

| | |
|---|---|
| Phenomenex Synergi Polar RP C₁₈, 4 μm | 2.0 × 50 mm |
| Temperature: | Ambient |

MS DetectorAcquisition Mode

| | |
|---|---|
| Applied Biosystem API4000 | under ESI positive mode |

Half Life Calculation (t_{1/2})

In the calculation of half life, we assumed that the disappearance of the mutual prodrug of this invention followed first order kinetics. Therefore,

C=C₀e^{-kt}

ln C=ln C₀−kt

The area peak ratio of prodrug vs IS was plotted against time first; the peak area ratios of later time points were normalized with the peak area ratio of initial time point (ASAP). The natural log of the normalized ratio was then plotted against time to generate a linear curve. The slope of this linear curve k was used for the following calculation.

Graphic plotted rate constant of loss K

At t_{1/2}, C₀=2C

t_{1/2}=ln 2/k

Drug Concentration Determination

Drug concentrations are calculated by normalizing the peak area ration to (t 0). Thus, calculated drug concentrations at any time point=normalized peak area ratio [t (0) mean/t mean]×initial drug concentration. Data (normalized peak area ratio) for the calculations of drug concentrations are listed in Table 1 and 2 for compounds in Example 29 (GS343071), salmeterol, and the dipeptide (M3 antagonist prepared by Sagara 2006).

TABLE 1

| | Example 29 (GS- | | Formation of | | Formation of dipeptide | |
|---|---|---|---|---|---|---|
| | 343071) | | Salmeterol | | (Sagara | |
| | in ALP (Peak | | in ALP (Peak | | 2006) in ALP | |
| | Area Ratio) | | Area Ratio) | | (Peak Area Ratio) | |
| Time (mins) | mean | Normalized | mean | Normalized | mean | Normalized |
| 0 | 0.2030 | 1.0000 | 0.0183 | 1.0000 | 0.1765 | 1.0000 |
| 15.0 | 0.1195 | 0.5887 | 0.0371 | 2.0273 | 0.3500 | 1.9830 |
| 30.0 | 0.0881 | 0.4340 | 0.0531 | 2.8989 | 0.4830 | 2.7365 |
| 45.0 | 0.0673 | 0.3313 | 0.0589 | 3.2186 | 0.4940 | 2.7989 |
| 60.0 | 0.0579 | 0.2850 | 0.0709 | 3.8716 | 0.5400 | 3.0595 |
| 75.0 | 0.0485 | 0.2387 | 0.0748 | 4.0874 | 0.5685 | 3.2210 |
| 90.0 | 0.0422 | 0.2079 | 0.0896 | 4.8934 | 0.6425 | 3.6402 |
| 105 | 0.0410 | 0.2017 | 0.0964 | 5.2650 | 0.6815 | 3.8612 |
| 120 | 0.0363 | 0.1788 | 0.0991 | 5.4153 | 0.6920 | 3.9207 |
| 135 | 0.0334 | 0.1643 | 0.1075 | 5.8743 | 0.7200 | 4.0793 |
| 150 | 0.0284 | 0.1397 | 0.1170 | 6.3934 | 0.7570 | 4.2890 |
| 165 | 0.0266 | 0.1310 | 0.1240 | 6.7760 | 0.7800 | 4.4193 |
| 180 | 0.0279 | 0.1372 | 0.1255 | 6.8579 | 0.8310 | 4.7082 |
| 210 | 0.0236 | 0.1163 | 0.1360 | 7.4317 | 0.8865 | 5.0227 |
| 240 | 0.0199 | 0.0978 | 0.1375 | 7.5137 | 0.8925 | 5.0567 |
| 270 | 0.0184 | 0.0906 | 0.1500 | 8.1967 | 1.0060 | 5.6997 |

TABLE 2

| | Added | | | | |
|---|---|---|---|---|---|
| | Compound | Calculated | | | |
| | Initial | Compound | Half | ALP | |
| | Conc. (μM) | Final Conc. | Life | Enzyme | Half Life |
| Com- | in Reaction | (μM) at | t_{1/2} | Conc. | in Buffer |
| pound | Mixture | 270 mins. | (mins) | (mg/mL) | Only |
| Exam- | | | 95.0 | 0.224 | |
| ple 29 | | | | | |
| | | | 62.4 | 0.224 | |
| | 86.6 | 7.8 | 43.2 | 0.443 | 770.2 mins |

1. A compound of the formula A 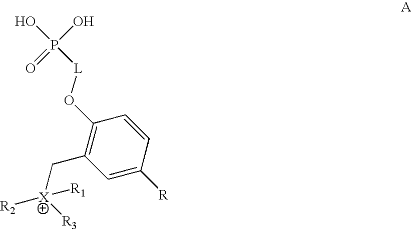
and pharmaceutical acceptable salts thereof, wherein:
X represents a quaternizable moiety;
R₁R₂R₃X taken together represent a muscarinic receptor antagonist (MRA) or its prodrug linking the parent MRA molecule to X;
L is either a bond or a methyleneoxy-(CH₂O) group; and
R is 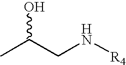
where R₄ is an alkyl group of 1-12 carbon atoms, arylalkyl or substituted arylalkyl where 1-3 CH₂ groups in the carbon chain may be replaced by atom(s) selected from O, S and NR₅ where R₅ is hydrogen or alkyl.

2. The compound of claim 1 wherein the MRA is M₃ selective.

3. The compound of claim 1 wherein the prodrug linking the parent MRA molecule to X is an acetyl ester.

4. The compound of claim 1 wherein L is a bond.

5. The compound of claim 1 wherein R₄ is (CH₂)₆O(CH₂)₄Ph or tert-butyl.

6. A compound as in claim 1 wherein
R is 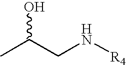
where
R₄ is (CH₂)₆O(CH₂)₄Ph or tert-butyl,
L is a bond, and
R₁R₂R₃X is selected from the group consisting of:
1-{4-Hydroxy-1-[3,3,3-tris-(4-fluoro-phenyl)-propionyl]-pyrrolidine-2-carbonyl}-pyrrolidine-2-carboxylic acid (1-methyl-piperidin-4-ylmethyl)-amide;
3-[3-(2-Diethylamino-acetoxy)-2-phenyl-propionyloxy]-8-isopropyl-8-methyl-8-azonia-bicyclo[3.2.1]octane (Ipratropium-N,N-diethylglycinate);
1-Cyclohexyl-3,4-dihydro-1H-isoquinoline-2-carboxylic acid 1-aza-bicyclo[2.2.2]oct-3-yl ester (Solifenacin);
2-Hydroxymethyl-4-methanesulfinyl-2-phenyl-butyric acid 1-aza-bicyclo[2.2.2]oct-3-yl ester (Revatropate);
2-{1-[2-(2,3-Dihydro-benzofuran-5-yl)-ethyl]-pyrrolidin-3-yl}-2,2-diphenyl-acetamide (Darifenacin);
4-Azepan-1-yl-2,2-diphenyl-butyramide (Buzepide);
7-[3-(2-Diethylamino-acetoxy)-2-phenyl-propionyloxy]-9-ethyl-9-methyl-3-oxa-9-azonia-tricyclo[3.3.1.02,4]nonane (Oxitropium-N,N-diethylglycinate);
7-[2-(2-Diethylamino-acetoxy)-2,2-di-thiophen-2-yl-acetoxy]-9,9-dimethyl-3-oxa-9-azonia-tricyclo[3.3.1.02,4]nonane (Tiotropium-N,N-diethylglycinate);
Dimethylamino-acetic acid 2-(3-diisopropylamino-1-phenyl-propyl)-4-methyl-phenyl ester (Tolterodine-N,N-dimethylglycinate);
3-[4,4-Bis-(4-fluoro-phenyl)-2-oxo-imidazolidin-1-yl]-1-methyl-1-(2-oxo-2-pyridin-2-yl-ethyl)-pyrrolidinium;
1-[1-(3-Fluoro-benzyl)-piperidin-4-yl]-4,4-bis-(4-fluoro-phenyl)-imidazolidin-2-one;
1-Cyclooctyl-3-(3-methoxy-1-aza-bicyclo[2.2.2]oct-3-yl)-1-phenyl-prop-2-yn-1-ol;
3-[2-(2-Diethylamino-acetoxy)-2,2-di-thiophen-2-yl-acetoxy]-1-(3-phenoxy-propyl)-1-azonia-bicyclo[2.2.2]octane (Aclidinium-N,N-diethylglycinate); and
(2-Diethylamino-acetoxy)-di-thiophen-2-yl-acetic acid 1-methyl-1-(2-phenoxy-ethyl)-piperidin-4-yl ester.

7. A compound as in claim 1 of formula B 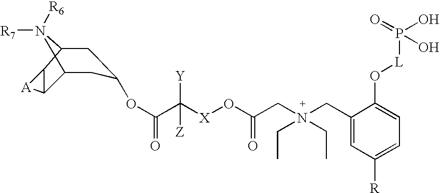
where L is a bond or CH₂—O;
R is 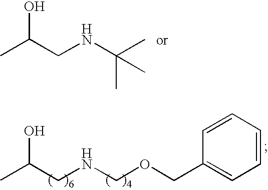
X is a bond or CH₂;
Y and Z are independently phenyl, 2-thienyl, or H;
R₆ is CH₃;
R₇ is ethyl, methyl or isopropyl; and
A is a bond or O.

8. A compound as in claim 1 of formula C 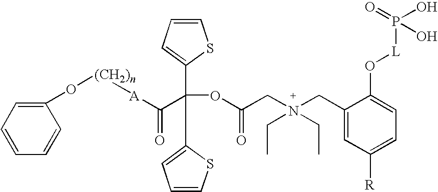
where L is a bond or CH₂—O;
R is 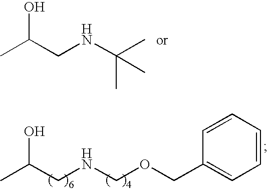
A is 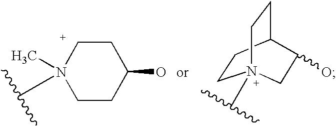
and
n is 2 or 3.

9. A compound as in claim 1 selected from the group consisting of:
Monophosphate of 3-(2-Hydroxymethyl-4-methanesulfinyl-2-phenyl-butyryloxy)-1-(5-{1-hydroxy-2-[6-(4-phenyl-butoxy)-hexylamino]-ethyl}-2-phosphonooxy-benzyl)-1-azonia-bicyclo[2.2.2]octane;
Monophosphate of (2-methylene-4-{1-hydroxy-2-[6-(4-phenyl-butoxy)-hexylamino]-ethyl}-phenyl)-3-[3-(2-Diethylamino-acetoxy)-2-phenyl-propionyloxy]-8-isopropyl-8-methyl-8-azonia-bicyclo[3.2.1]octane; and
Monophosphate of 3-(1-Cyclohexyl-3,4-dihydro-1H-isoquinoline-2-carbonyloxy)-1-(5-{1-hydroxy-2-[6-(4-phenyl-butoxy)-hexylamino]-ethyl}-2-phosphonooxy-benzyl)-1-azonia-bicyclo[2.2.2]octane.

10. (canceled)

11. An aerosol formulation for the prevention and treatment of pulmonary bronchoconstriction, said formulation comprising from about 10 μg to about 1000 μg of at least one monophosphate mutual prodrug as in claim 1, wherein said formulation is adapted to be administered by aerosolization to produce predominantly aerosol particles between 1 and 5μ.

12. An aerosol formulation as in claim 11, wherein the mutual prodrug is prepared as a dry powder and the formulation is administered using a dry powder inhaler.

13. (canceled)

14. An aerosol formulation for the prevention and treatment of pulmonary bronchoconstriction, said formulation comprising from about 10 μg to about 1000 μg of at least one mutual prodrug as in claim 1, prepared as a dry powder for aerosol delivery in a physiologically compatible and tolerable matrix, wherein said formulation is adapted to be administered using a dry powder inhaler able to produce predominantly aerosol particles between 1 and 5μ.

15. A method for the prevention and treatment of pulmonary bronchoconstriction, comprising administering to a patient in need of such treatment an effective amount of an aerosol formulation comprising about 10 μg to about 1000 μg of at least one mutual prodrug as in claim 1.

16. A method as in claim 15 wherein when the mutual prodrug is delivered to the lung, the phosphate group is cleaved by an endogenous enzyme (alternatively followed by the action of an endogenous esterase) and the MRA and the β-agonist are individually released in a simultaneous manner.

17. (canceled)
